(19) United States
(12) Patent Application Publication
(10) Pub. No.: US 2010/0114148 A1
(43) Pub. Date: May 6, 2010

(54) DEVICES AND METHODS FOR ADJUSTING A SATIATION AND SATIETY-INDUCING IMPLANTED DEVICE

(76) Inventors: Thomas E. Albrecht, Cincinnati, OH (US); Jason L. Harris, Mason, OH (US); Mark S. Ortiz, Milford, OH (US); Amy L. Marcotte, Mason, OH (US); Daniel F. Dlugos, JR., Middletown, OH (US); Mark S. Zeiner, Mason, OH (US); Michael J. Stokes, Cincinnati, OH (US); Jeremy D. Jarrett, Cincinnati, OH (US); Matthew D. Daniel, Lebanon, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,078

(22) Filed: Oct. 30, 2008

(51) Int. Cl. A61M 29/02

(52) U.S. Cl. 606/192

(57) ABSTRACT

An implant for placement within a hollow body organ having a member with an undeployed shape, for delivery within a hollow body, and one or more deployed shapes, for implantation therein. The member has sufficient rigidity in its deployed shape to exert an outward force against an interior of the hollow body so as to bring together two substantially opposing surfaces of the hollow body. The implant also includes a means for changing the deployed shape of the member while implanted within the hollow body.

This case is related to the following commonly assigned and concurrently filed U.S. Applications, all of which are hereby incorporated herein by reference:

U.S. Ser. No. ______ (Attorney Docket Number END6514USNP) titled DEVICES and METHODS FOR ADJUSTING A SATIATION AND SATIETY-INDUCING IMPLANTED DEVICE; U.S. Ser. No. ______ (Attorney Docket Number END6515USNP) titled Sensor Trigger; U.S. Ser. No. ______ (Attorney Docket Number END6516USNP) titled AUTOMATICALLY ADJUSTING INTRA-GASTRIC SATIATION AND SATIETY CREATION DEVICE; U.S. Ser. No. ______ (Attorney Docket Number END6517USNP) titled OPTIMIZING THE OPERATION OF AN INTRA-GASTRIC SATIETY CREATION DEVICE; U.S. Ser. No. ______ (Attorney Docket Number END6518USNP) titled POWERING IMPLANTABLE DISTENSION SYSTEMS USING INTERNAL ENERGY HARVESTING MEANS; U.S. Ser. No. ______ (Attorney Docket Number END6519USNP) titled WEARABLE ELEMENTS FOR INTRA-GASTRIC SATIETY CREATION SYSTEMS; U.S. Ser. No. ______ (Attorney Docket Number END6520USNP) titled INTRA-GASTRIC SATIETY CREATION DEVICE WITH DATA HANDLING DEVICES AND METHODS; U.S. Ser. No. ______ (Attorney Docket Number END6521USNP) titled GUI FOR AN IMPLANTABLE DISTENSION DEVICE AND A DATA LOGGER; U.S. Ser. No. ______ (Attorney Docket Number END6522USNP) titled METHODS AND DEVICES FOR FIXING ANTENNA ORIENTATION IN AN INTRA-GASTRIC SATIETY CREATION SYSTEM; U.S. Ser. No. ______ (Attorney Docket Number END6523USNP) titled METHODS AND DEVICES FOR PREDICTING INTRA-GASTRIC SATIETY CREATION DEVICE SYSTEM PERFORMANCE; U.S. Ser. No. ______ (Attorney Docket Number END6524USNP) titled CONSTANT FORCE MECHANISMS for Regulating Distension Devices; U.S. Ser. No. ______ (Attorney Docket Number END6525USNP) titled A METHOD OF REMOTELY ADJUSTING A SATIATION AND SATIETY-INDUCING IMPLANTED DEVICE.

### FIELD OF THE INVENTION

The present invention relates generally to obesity treatment and, more particularly, to the treatment of obesity by implanting a force producing device into a gastric lumen to create pressure on the inside surface of the lumen to reduce the effective volume of the lumen inducing a prolonged sense of satiety and/or and earlier feeling of satiation in the patient, and adjusting the device remotely or automatically.

### BACKGROUND OF THE INVENTION

Obesity is a medical condition affecting more than 30% of the population in the United States. Obesity affects an individual's personal quality of life and contributes significantly to morbidity and mortality. Obesity is most commonly defined by body mass index (BMI), a measure which takes into account a person's weight and height to gauge total body fat. It is a simple, rapid, and inexpensive measure that correlates both with morbidity and mortality. Overweight is defined as a BMI of 25 to 29.9 kg/m² and obesity as a BMI of ≧30 kg/m². Morbid obesity is defined as BMI≧40 kg/m² or being 100 lbs. overweight. Obesity and its co-morbidities are estimated to cost an excess of $100 billion dollars annually in direct and indirect health care costs. Among the co-morbid conditions which have been associated with obesity are type 2 diabetes mellitus, cardiovascular disease, hypertension, dyslipidemias, gastroesophageal reflux disease, obstructive sleep apnea, urinary incontinence, infertility, osteoarthritis of the weight-bearing joints, and some cancers. These complications can affect all systems of the body, and dispel the misconception that obesity is merely a cosmetic problem. Studies have shown that conservative treatment with diet and exercise alone may be ineffective for reducing excess body weight in many patients.

Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure, a small stomach pouch is separated from the remainder of the gastric cavity and attached to a resected portion of the small intestine.

This resected portion of the small intestine is connected between the “smaller” gastric pouch and a distal section of small intestine allowing the passage of food therebetween. The conventional RYGB procedure requires a great deal of operative time and is not without procedure related risks. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing an early feeling of satiation and/or a prolonged feeling of satiety. A band is placed around the stomach near the junction of the stomach and esophagus. The small upper stomach pouch is filled quickly, and slowly empties through the narrow outlet to produce the feelings of satiety and satiation. In addition to surgical complications, patients undergoing a gastric banding procedure may suffer from esophageal injury, spleen injury, band slippage, band erosions, reservoir deflation/leak, and persistent vomiting. Other forms of bariatric surgery that have been developed to treat obesity include Fobi pouch, bilio-pancreatic diversion, vertical banded gastroplasty and sleeve gastrectomy. As aspects of some of these procedures, including RYGB, involve stapling a portion of the stomach, many bariatric procedures are commonly referred to as “stomach stapling” procedures.

For morbidly obese individuals, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss. Further, it is known that modest reductions in weight may significantly decrease the impact of co-morbid conditions including, but not limited to type 2 diabetes mellitus. For this reason as well, a low cost, low risk procedure with effective weight loss results would provide significant benefit to both patients and health care providers.

Accordingly, it is desirable to have a low risk, minimally invasive procedure for treating obesity. It is desirable to have a procedure in which a treatment device can be easily and safely implanted into the gastric cavity of a patient to reduce the effective volume of the cavity. Additionally, it is desirable to have such a device that can assume an initial deploying configuration, and then be transformed into a second operable configuration within the gastric cavity. Further, it is desirable that the device apply outward pressure against the wall of the gastric cavity in the operable configuration in order to create a sensation of fullness within the patient. Further, it is desirable to have a method of treating obesity by reducing the effective volume within the gastric cavity. Additionally, it is desirable to have a method of treating obesity which includes applying pressure against the inside surface of the gastric cavity to create a feeling of fullness. It is desirable that the obesity treatment method be low cost and minimally invasive so as to be beneficial to a large number of obese patients. Further, it is desirable that the obesity treatment be easily and safely reversible. Additionally, it is desirable to adjust the device over time to accommodate individual patients and extend the durability of the treatment effect. The present invention provides an implantable obesity treatment device and method of treating obesity which achieves these objectives.

### BRIEF DESCRIPTION OF THE DRAWINGS

FIG. 1 is a perspective view of an open-loop, U-shaped satiety and satiation inducing coil;

FIG. 2 is a schematic view illustrating an external port for remotely adjusting an implanted coil;

FIG. 3 is a close-up view of a gastric lumen, taken from FIG. 2, showing a first embodiment for remotely adjusting the implanted coil;

FIG. 4 is a cross-sectional view taken along line B-B of FIG. 3, showing the adjustment wire of the first embodiment in greater detail;

FIG. 5 is a detailed view of the coil and adjustment wire of FIG. 4, showing offset positions for the adjustment wire;

FIG. 6 is a cross-sectional view of a gastric lumen showing a second embodiment for remotely adjusting an implanted coil;

FIG. 7 is a cross-sectional view similar to FIG. 6, showing the coil in an expanded position following the severing of a first suture connection;

FIG. 8 is a cross-sectional view of a gastric lumen showing a third embodiment for remotely adjusting an implanted coil;

FIG. 9A is a more detailed view of the suture and winch of FIG. 8;

FIG. 9B is a cross-sectional view taken along line C-C of FIG. 8, showing an adjustment device for the winch of FIG. 8;

FIG. 10 is a cross-sectional view of a gastric lumen showing a fourth embodiment for remotely adjusting an implanted coil;

FIG. 11 is a cross-sectional view of the coil of FIG. 10, taken along line D-D;

FIG. 12 is a cross-sectional view similar to FIG. 10, showing the coil being activated by a light source;

FIG. 13 is a cross-sectional view of a gastric lumen showing a fifth embodiment for remotely adjusting an implanted coil;

FIG. 14 is a cross-sectional view taken along line E-E of FIG. 13;

FIG. 15 is a cross-sectional view of a gastric lumen showing a sixth embodiment for remotely adjusting an implanted coil;

FIG. 16 is a cross-sectional view taken along line F-F of FIG. 15, showing a device for changing the fluid level within the implanted coil;

FIG. 17 is a cross-sectional view of the gastric lumen showing a 7^{th} embodiment for an adjustable implanted coil;

FIG. 18 is a cross-sectional view of a gastric coil device;

FIG. 19 is a detail view of individual links of a gastric coil device;

FIG. 20 is a detail view of individual links of a gastric coil device;

FIG. 21 is a detailed cross-sectional view of the individual links of a gastric coil device;

FIG. 22 is a cross-sectional view of a gastric coil device being filled with a fluid;

FIG. 23 is a cross-sectional view of a gastric coil device being fully expanded;

FIG. 24 is a cross-sectional view of a gastric lumen with the gastric coil device being fully expanded; and

FIG. 25 is a side view of a gastric lumen being acted upon by the gastric coil device.

### DETAILED DESCRIPTION OF THE INVENTION

The present invention pertains to devices and methods for remotely adjusting a satiety-inducing gastric implant. The implant is shaped as an open loop within a gastric lumen to hold the walls of the lumen taut, thereby reducing the effective volume per tissue surface area within the lumen. The stretching of the stomach tissue inhibits gastric motility and delays gastric emptying. When utilized within the stomach, the implant can induce a prolonged hormonal response within the body to reduce the desire to eat. In addition, the implant can bias stretch receptors within the stomach to signal an early sense of satiety. The implant is shaped as a semi-rigid open loop (e.g., U-shaped, V-shaped, etc.) that applies an outward radial force in a single plane to flatten the stomach. Alternatively, the device may be shaped as a closed loop (e.g., O-Shaped, D-Shaped, ‘figure eight’ shaped, etc.). One example is a D shape in which the straight line of the D is hingedly attached to the loop of the curved part of the D. The device would be inserted either collapsed or straight, then formed into a closed loop. These loop features are important to the implant as they prevent the implant from exiting the gastric region. The implant has a minimum bend radius in the loop region to prevent it from passing through the pylorus and or esophagus (proximal or distal migration) where the coil device has a mechanical stop that prevents it from folding any smaller than the minimum bend radii. The force plane of the implant may change over time due to stomach motions. The implant is preferably installed and removed endoscopically and includes design features that prevent migration and erosion. Although less desirable, laparoscopic, open surgical techniques, or a combination thereof, may also be used to install and remove the implant. Examples of satiety-inducing gastric implants, optimal design features, as well as methods for installing and removing them are described in commonly owned and pending U.S. patent application Ser. No. 11/469,564, filed Sep. 1, 2006, and pending U.S. patent application Ser. No. 11/469,562, filed Sep. 1, 2006, which are hereby incorporated herein by reference in their entirety.

The present invention provides an implantable device for placement within a hollow body organ having an undeployed shape for delivery to the hollow body organ and a deployed shape within the hollow body organ. The device includes tensioning members for drawing and locking the ends of the device in a curved, deployed shape. Retained forces within the deployed device produce outward pressure on the hollow body organ to flatten the organ in the plane of the device, thereby reducing the effective volume within the organ.

The present invention also provides a method for treating obesity which includes passing an implantable device into a hollow body organ in an undeployed configuration and placing the device into a deployed configuration within the hollow body organ. The method includes means for remotely adjusting the device in order to optimize treatment effect. In the curved configuration, pressure is applied by the device against the interior of the hollow body organ, to flatten the organ in the plane of the device, thereby reducing the volume within the organ.

The device may have several effects on the stomach resultant from its action. The distension of the stomach at all times or periodically may trigger stretch receptors which in turn trigger a sense of satiation. At the same time, the device may limit the stomach's ability to expand, effectively reducing its capacity or fill volume. Additionally, the device may induce a hormonal effect due either to the triggering of the stretch receptors, contacting of various nerves on the stomach or other mechanism. Additionally, the device may prolong gastric emptying by preventing efficient antral contractions and/or partially blocking the pyloric outlet. Additionally, the device may provide a restrictive inlet into the stomach just distal to the esophagogastric junction. All of these may impact satiation (level of fullness during a meal, which regulates the amount of food consumed), or satiety (level of hunger after a meal is consumed, which regulates the frequency of eating.

The implant is preferably inserted trans-orally while deformed, folded, or otherwise placed into a relatively straight position, and is subsequently deployed into a different shape while inside the stomach lumen. The deployed configuration is flexible enough to allow some compression and movement relative to the stomach mucosa to prevent erosion, but rigid enough to prevent unwanted buckling and proximal or distal migration. The implant can be adjusted periodically to relieve the pressure on the gastric walls, or to alter the size or shape of the implant. The size, shape or stiffness of the implant may be adjusted multiple times after implantation as the patient acclimates to the device. The present invention provides for the remote accessing and adjustment of a satiety-maintaining and/or satiation inducing gastric implant. Remote access eliminates the need to intubate the patient endoscopically in order to adjust the implant.

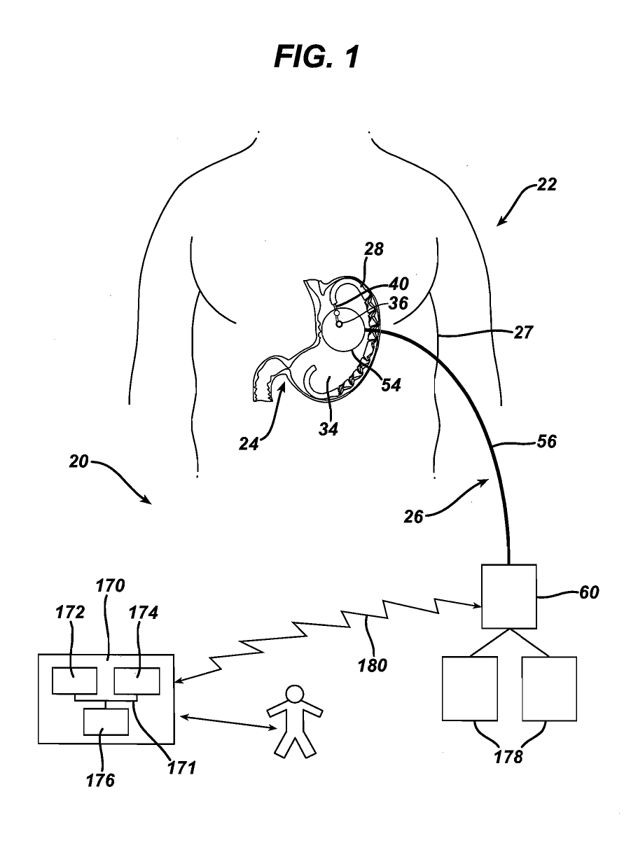

FIG. 1 illustrates an implant device that may be remotely adjusted in accordance with the present invention. In this embodiment, the implant device is an open loop coil 20. Coil 20 has a substantially straight configuration during deployment, and a curved, U-shaped configuration once deployed in the lumen. When deployed, coil 20 applies an outward force against the walls of the gastric lumen, keeping the tissue taut, but allowing enough flexibility to prevent damage to the stomach lining. Coil 20 comprises a slightly-curved atraumatic portion 22 and distal ends 24, 26 that curve inwardly at a smaller diameter than portion 22. Ends 24, 26 have sufficient stiffness and diameter to prevent proximal and distal migration of the coil after it is placed in the gastric lumen. Ends 24, 26 also provide blunt ends to prevent damage of the stomach lining. A plurality of peaks 30 and valleys 32 along the outer surface of coil 20 provide a pathway for the gastric content to travel through to the pylorus, thereby slowing gastric emptying yet preventing a complete obstruction. Peaks 30 and valleys 32 also prevent the erosion of coil 20 into the gastric tissue, by providing a gripping surface for the stomach's peristaltic movements to propel the device in a circular motion, as indicated by reference numeral 34. Each of the coil ends 24, 26 include a hole 36, 40 to aid in the removal and adjustment of coil 20. Holes 36, 40 provide an attachment location for a suture, wire or other flexible material. Additionally, a lumen 42 extends substantially through the length of coil 20. Lumen 42 is designed to accept a stiffening or movable element to allow for adjustment of the coil.

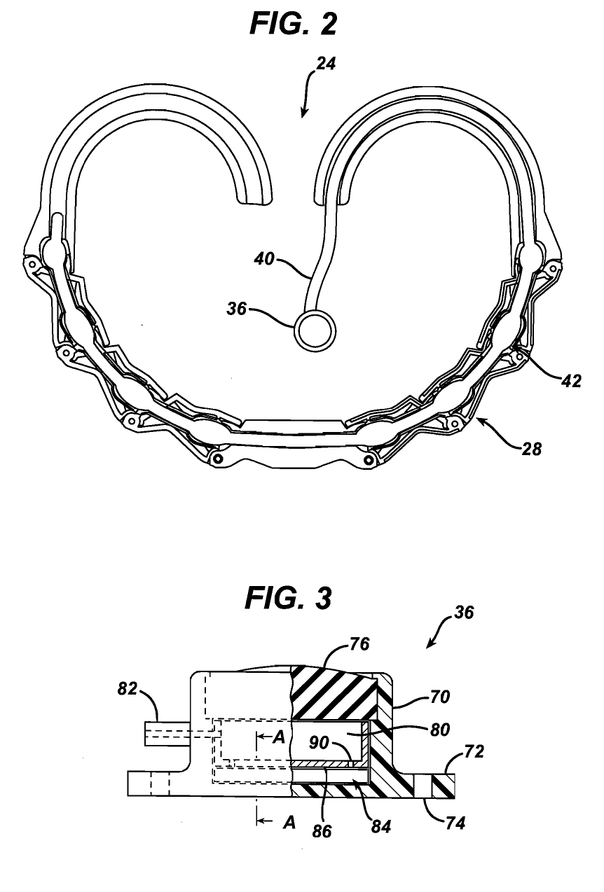

Referring now to FIGS. 2-4, to remotely adjust coil 20 a port 44 is inserted through an abdominal wall 50 of the patient and into the interior of a gastric cavity 46. Port 44 is preferably a PEG tube that is sized to allow mechanical, electrical or fluid communication with the implanted coil. Port 44 may be implanted prior to, during or after the implantation of coil 20. For a one time adjustment of coil 20, port 44 may be inserted and removed during a single procedure. Alternatively, port 44 may be inserted and retained within the abdominal wall in a semi-permanent manner to allow for multiple adjustments of the implant over a period of time.

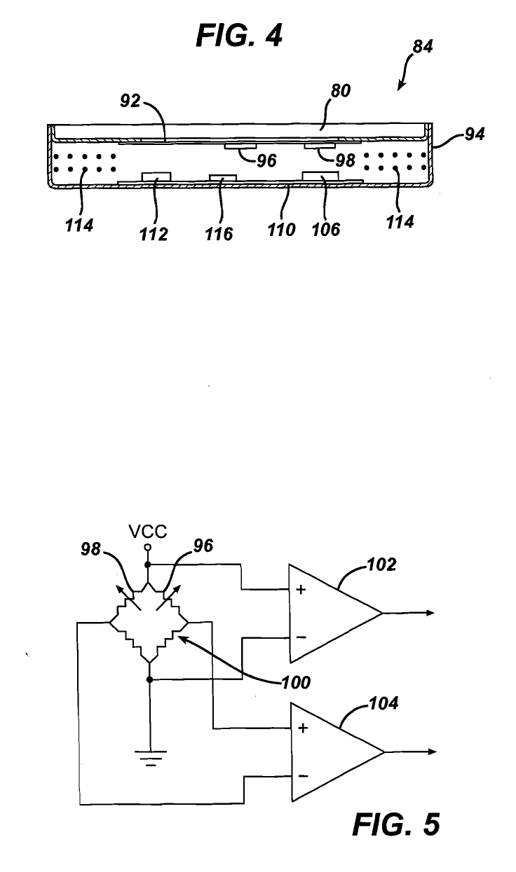

FIG. 3 shows a first embodiment for adjusting coil 20 through port 44. In this embodiment, an adjustment wire 52 is housed within a sheath 54. The adjustment wire and sheath extend through lumen 42 of coil 20. Wire 52 is securely attached at one opening of lumen 42 to one of the distal ends 24, 26 of coil 20. The other, free end of wire 52 extends through sheath 54 outside the opposite opening of lumen 42. To adjust coil 20, a knob 56 is disposed on the external side of port 44, as shown in FIG. 4. The free end of wire 52 extends through port 44 and is attached to knob 56. Wire 52 may be pulled externally through sheath 54 by turning knob 56 in a first direction. As knob 56 turns, pulling on wire 52, the wire tension is passed back into lumen 42 to the attached end of the wire. The tension on wire 52 shortens the length of the wire within lumen 42. When adjustment wire 52 is offset from the bending moment of coil 20 towards the inner diameter 110 of the coil, as shown in FIG. 5, pulling on the wire draws the ends 24, 26 of coil 20 closer together. As the coil ends are drawn together, the diameter of coil 20 decreases, thereby reducing the force of the implant against the gastric walls. Alternatively, when adjustment wire 52 is offset from the bending moment of coil 20 towards the outer diameter 111 of the coil, as shown by the dashed lines 58 in FIG. 5, pulling on the wire causes ends 24, 26 of coil 20 to move apart, thereby expanding the size of the coil and increasing the force against the gastric cavity walls.

The length of wire 52 within coil lumen 42 may be increased by turning knob 56 in an opposite direction to push on the wire. As wire 52 is pushed, an additional length of wire 52 is forced through sheath 54 and into lumen 42. When wire 52 is offset towards the inner diameter 110 of coil 20, pushing additional wire into lumen 42 allows the ends 24, 26 of the coil to move apart. As ends 24, 26 move apart, coil 20 expands, increasing the coil's force against the gastric walls. Likewise, when wire 52 is offset towards the outer diameter 111 of coil 20, pushing additional wire into lumen 42 will reduce the tension within coil 20, allowing ends 24, 26 of the coil to move closer together, thereby reducing the diameter of the coil. After wire 52 is adjusted, knob 56 is locked into position to secure the wire and prevent a subsequent change in wire tension.

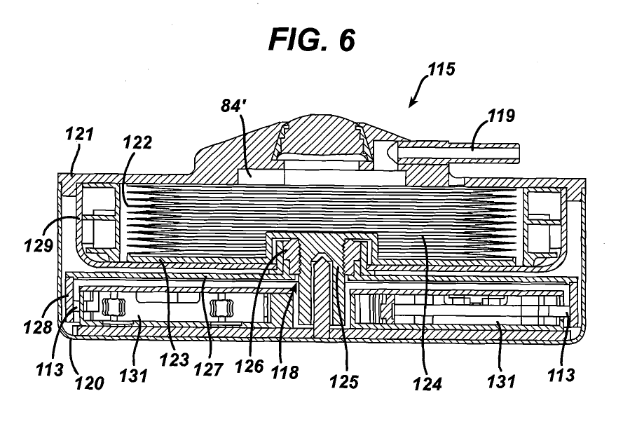

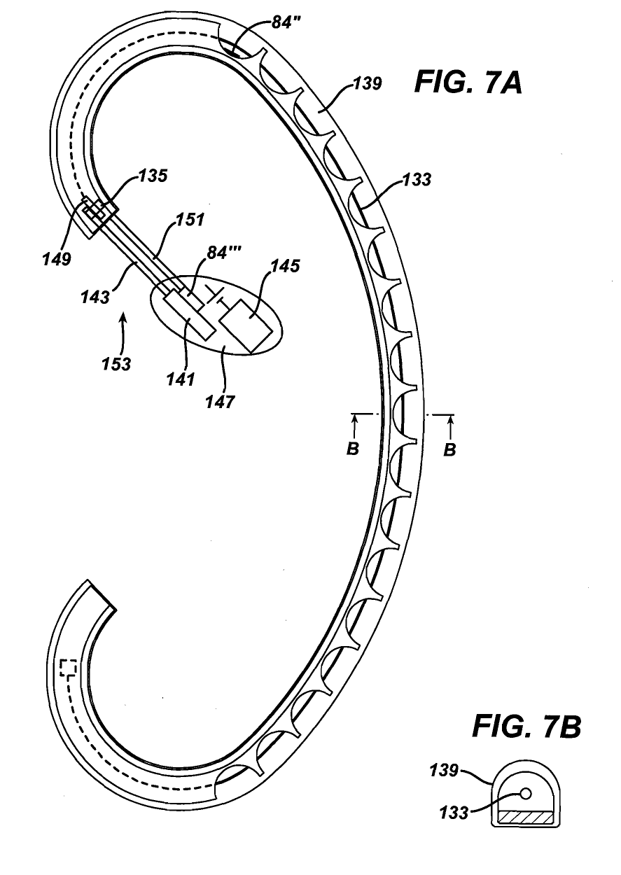

FIG. 6 shows a second embodiment for remotely adjusting an implanted coil 20 through port 44. In this embodiment, a plurality of pieces of flexible material 60 extend through the open loop of coil 20. Flexible material 60 may, for example, be strands of suture material. Each of the suture strands 60 is attached at one end to hole 36 and at the opposite end to hole 40 to constrain coil 20 in a compressed configuration. Suture strands 60 have differing lengths to create different degrees of compression within coil 20. Initially, the shortest piece of suture is held tautly between the coil ends to compress the coil into a minimum size. To expand the size and shape of coil 20, a tool may be passed through port 44 to selectively sever one or more of the sutures 60. Alternatively, a tool may be passed trans-esophageally to cut sutures 60. The sutures are preferably severed in the order of increasing length, with the shortest suture cut first, to gradually expand the size of the coil. As each suture is cut, coil 20 expands until the next shortest suture is taut between coil ends 24, 26. FIG. 7 shows coil 20 in an expanded shape after the shortest one of the sutures 60 has been cut, and the next shortest suture is held tautly between coil ends 24, 26. Any number of sutures 60 may be cut until coil 20 obtains the desired size. Multiple sutures may be cut in a single adjustment procedure. Alternatively, individual sutures may be cut over a period of time to gradually expand the size of coil 20. Sutures may have distinguishing features such as color to aid in the identification of a desired target for cutting.

In an alternative embodiment, sutures 60 may be comprised of a plurality of bioabsorbable materials that gradually degrade over time. The degradation period of the materials can vary between the various suture pieces, so that the sutures dissolve at different rates. The degradation rate can be controlled by changes in cross sectional area or in degradation rates of the material for example. The length of the degradation period corresponds to the length of the suture piece, so that the pieces dissolve in the order of increasing length to gradually expand the size of the coil. In this embodiment, coil expansion can be accomplished without penetrating the gastric cavity through port 44.

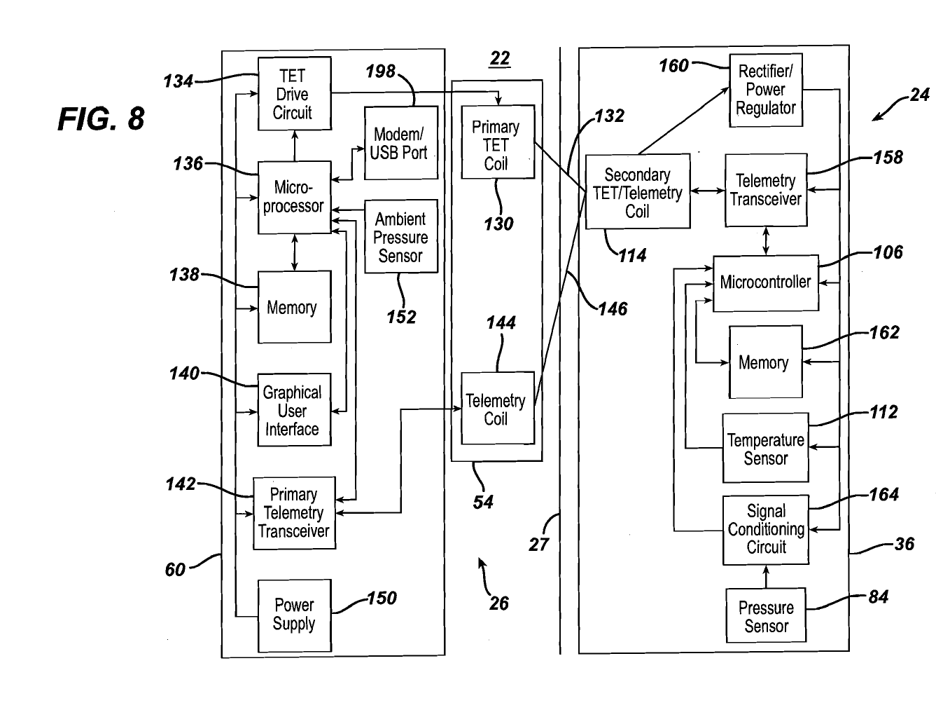

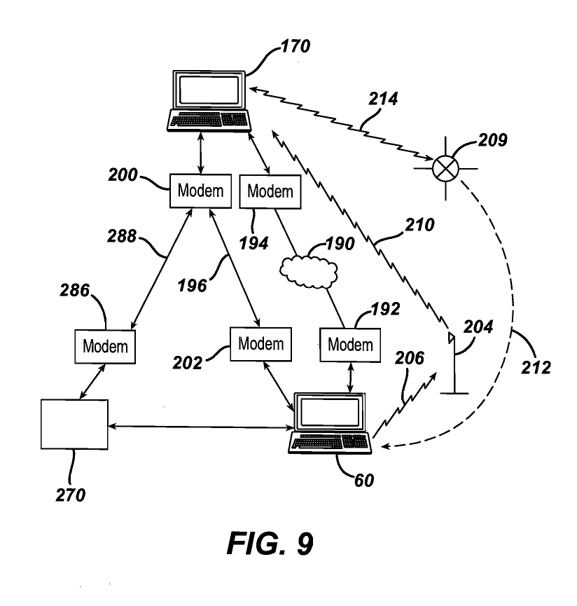

FIG. 8 shows a third embodiment for remotely adjusting a coil 20 through port 44. In this embodiment, a winch 62 is embedded in one of the coil ends 24, 26. A first end of a suture material 64 is wound around winch 62, as shown in greater detail in FIG. 9A. A second end of the suture is securely attached to the other one of the coil ends 24, 26. Suture 64 is held between the coil end and winch 62 to maintain coil 20 in a compressed configuration. To expand the size of coil 20, winch 62 is turned in a first direction to increase the suture length between the winch and coil end. Alternatively, to reduce the size of coil 20, winch 62 is turned in a second direction to reduce the suture length and draw coil ends 24, 26 closer together. As shown in FIG. 9B, a torsional cable 66 extends from winch 62 through port 44 to operate the winch. Cable 66 is housed within a sheath 68 and attached at an external end to knob 56. When knob 56 is turned, cable 66 moves within sheath 68 to turn winch 62, and thereby adjust the length of suture material 64 extending between the coil ends.

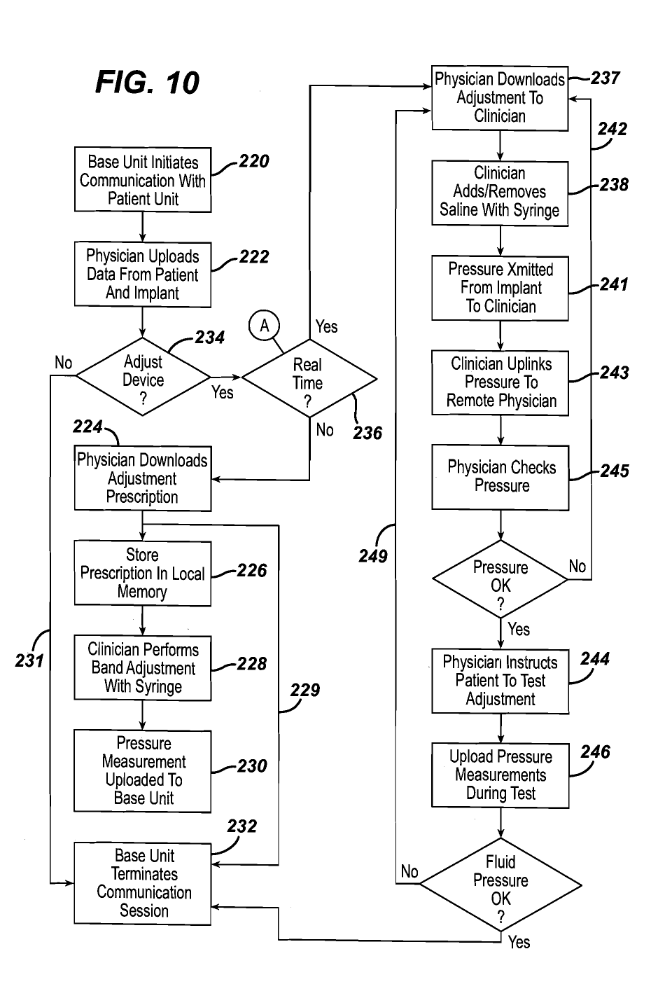

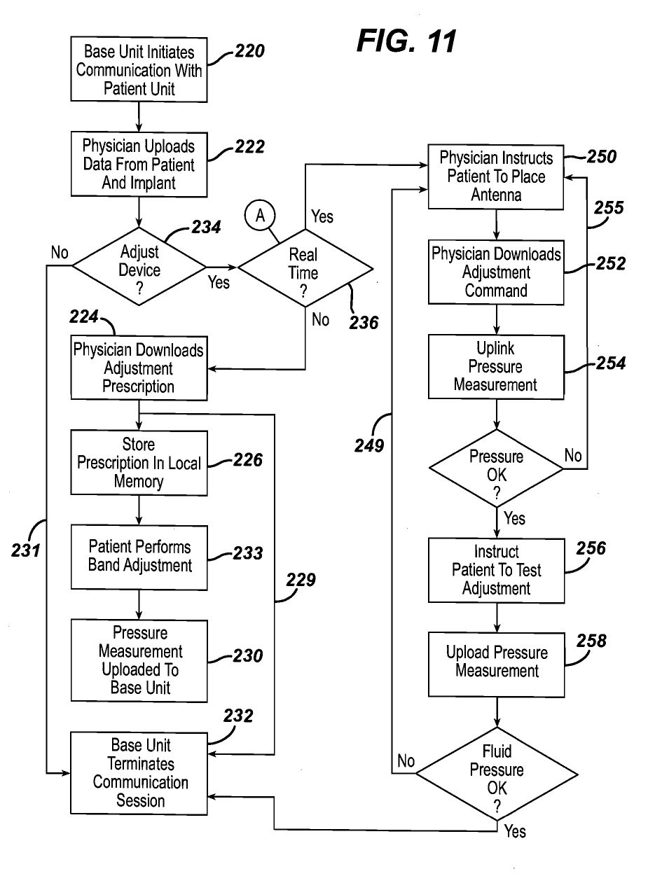

FIG. 10 shows yet another embodiment for remotely adjusting an implanted coil. In this embodiment, the implant is a coil 70 formed as a composite of multiple layers of a shape memory material. This shape memory material may, for example, be Nitinol, a nickel-titanium alloy. Changes in the shape of Nitinol can be induced by exposing it to a specific temperature. Other shape memory materials include polymers that are activated by exposure to an environmental element which effects a change in the shape of the polymer. The shape change in the polymer produces a corresponding change in the size or shape of coil 70. In the embodiment shown in FIGS. 10 and 11, coil 70 comprises three different polymer layers which each extend the circumferential length of the coil. Coil 70, however, can be made up of any number of different polymer layers depending upon the desired degree and rate of coil expansion. Each of the different polymer layers has a different activation point for expansion. The different activation points allow for successive activation of the layers, thereby producing a gradual change in the size of the coil. These layers may also be comprised of a bimetallic material so that when exposed to varying temperature the elements will expand or contract. Further, the elements may be comprised of bladders with a ferrofluidic material that changes shape (enlarges) in the presence of a magnetic field.

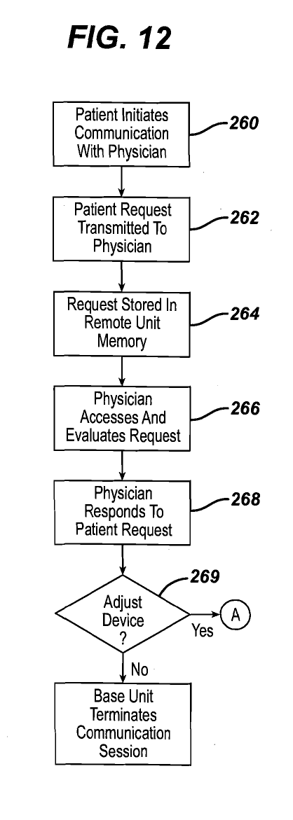

As shown in FIG. 12, one way in which the polymer layers in coil 70 may be activated is by exposure to specific light frequencies. In this embodiment, a light source 76 producing a beam of a specific frequency is passed into gastric cavity 46, either transesophageally through an endoscope, or through port 44 as shown. The light source is applied to coil 70 to activate one of the polymer layers. As the light beam penetrates coil 70, one of the layers within the coil expands to increase the diameter of the coil. Each of the different polymer layers may be activated by a different light frequency, so that a gradual expansion of coil 70 can be accomplished. In an alternative embodiment, coil 70 may be made up of shape memory polymers that are activated by exposure to a magnetic field. In this embodiment, the size of the coil may be changed by passing a current through port 44 to create a magnetic field adjacent to the coil. The magnetic field causes one or more of the polymer materials in the coil to expand, thereby changing the shape of the coil. In yet another embodiment, the shape memory polymers may be thermally activated to affect a change in the coil shape. A current may be selectively applied to heat one or more polymer layers until the desired coil shape is achieved. Additionally, a shape memory polymer that is sensitive to pH levels could be employed. When the pH level of the stomach is low as is the case during fasting, the device could be in a small configuration. As the pH level of the stomach raises during eating, the device would expand and send an early sense of satiation.

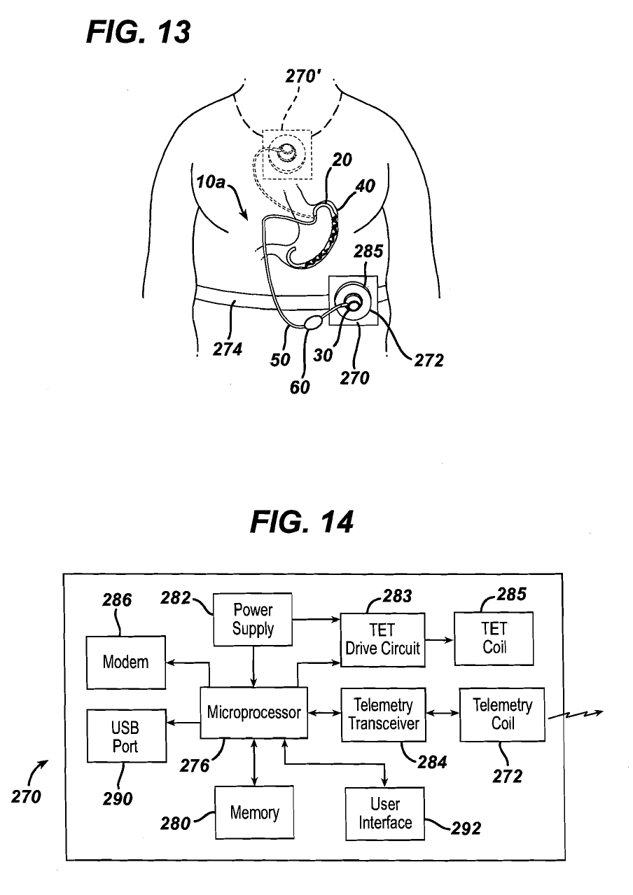

FIGS. 13 and 14 illustrate another embodiment for adjusting an implanted gastric satiety-enhancing or satiation inducing device. In this embodiment, the implant is an open loop coil 80 having an inner core 82 and an outer covering 84. Inner core 82 is composed of a stiffening material that is biased to expand outwardly into a maximum size and shape. An exemplary stiffening material for inner core 82 is Nitinol. Covering 84 is molded over inner core 82 to compress and constrain the inner core into a reduced diameter configuration. Covering 84 is comprised of one or more bioabsorbable materials that gradually erode off of the exterior of inner core 82 following implantation within the gastric cavity. Cover materials may erode at different rates. As covering 84 is slowly absorbed away from coil 80, the energy stored in compressed inner core 82 is released, allowing the coil to expand in size within the gastric cavity. When covering 84 is completely eroded off of inner core 82, coil 80 assumes a maximum size within the cavity. In this embodiment, coil 80 can be adjusted without outside intervention through port 44. The Nitinol may be preshaped such that the coil will expand with one segment eroding off and alternatively contracting with an additional segment eroding off. The central coil may also be made up of multiple segments of stiffening material such that, after implantation, the individual segments may be added or removed to control the forces exerted on the stomach.

In a slight modification to the embodiment shown in FIGS. 13 and 14, a stiffening material may be inserted into a coil to alter or constrain the shape of the coil. In this modification, the coil is comprised of a biodegradable material having a lumen extending the length of the coil. The stiffening member is inserted into the coil lumen prior to implantation. After implantation, the coil is slowly absorbed into the gastric cavity, leaving the stiffening member which expands and assumes a larger size within the cavity. An exemplary stiffening material for this alternative embodiment is Nitinol.

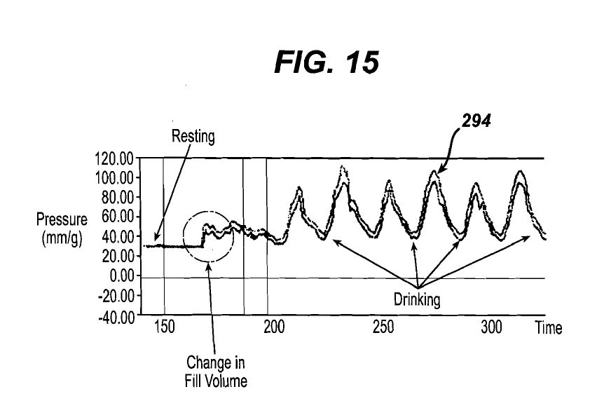

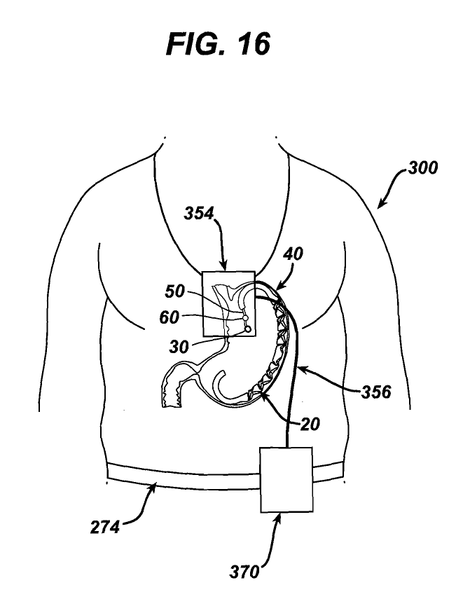

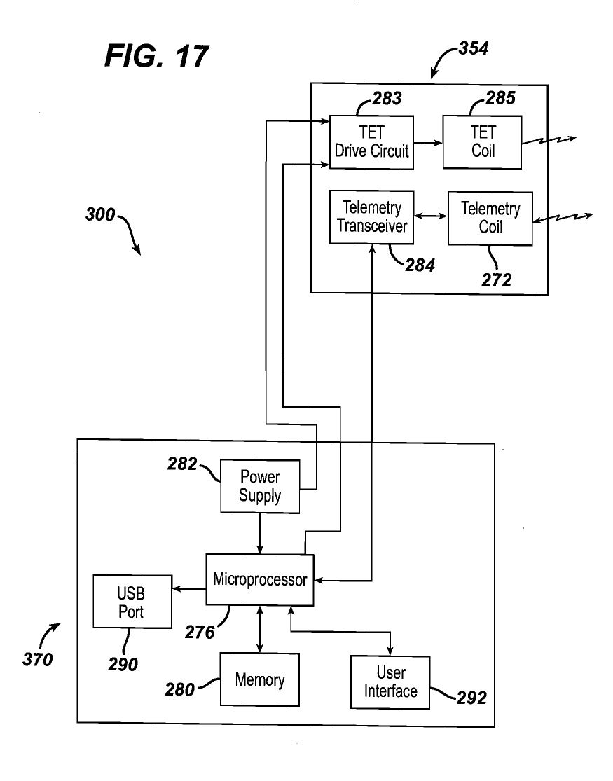

FIG. 15 illustrates yet another embodiment for adjusting an implanted gastric coil. In this embodiment, a coil 90 includes a plurality of fluid-filled sacs 92 spaced along the length of the coil. Each of the sacs 92 is in fluid communication with the other sacs through a channel 94 that extends the length of the coil. Fluid is injected into or removed from sacs 92 to vary the size of coil 90. Coil 90 is biased into a small diameter configuration when sacs 92 are empty. As fluid is injected into sacs 92 through channel 94, the volume of each sac expands, causing the sacs to force the coil to expand outwardly and increase the force of the coil against the gastric cavity walls. Likewise, when fluid is removed from sacs 92, the force against the rigid portion of coil 90 is decreased, allowing the coil to relax into a smaller diameter configuration. As shown in FIG. 16, fluid may be manually added to or removed from sacs 92 through a syringe 96 injected into channel 94 via a port 100. Alternatively, a fluid reservoir with a control valve may be connected to channel 94, and fluid periodically injected into or removed from the channel based upon a signal. As shown in FIG. 15, adding fluid to sacs 92 serves to expand, or straighten the coil as the sacs are spaced along or near the interior surface of the coil. By placing the sacs on or near the exterior surface of the coil (i.e., the surface applying the greatest pressure to the gastric wall) the device may be contracted in size or shape with the addition of fluid. Similar alterations may be made to the embodiments described below starting in FIG. 17.

A sensor 102 may be located within coil 90 to measure the fluid pressure within sacs 92 and channel 94. The signal from sensor 102 can be used to control fluid exchange with channel 94. In addition to fluid pressure, sensors may be located within or external to coil 90 to measure clinically relevant parameters such as conditions within the gastric cavity or from within the coil itself These measurements may be used to provide feedback either automatically or manually regarding operation of the coil. This feedback may be used to make adjustments to the coil. For example, a sensor could measure changes in the stomach pH, pressure, or internal device strains indicating that the patient was eating. The sensor could then generate a signal to increase the size of the coil in order to induce satiation sooner. When the readings returned to a previous level, indicating the patient was finished eating, the coil could be returned to a previous size. In one embodiment, this rate at which the coil returns to a previous size may be fast or slow, but is optimized to prolong feelings of satiety. A sensor within coil 90 could also be used to record operational data for the coil. This data could be retrieved from the coil during a medical checkup.

A sensor or array of sensors, not show, may be fixedly attached to the external surface of the coil to measure the interface pressure or force between the coil and stomach tissue. The feedback from these sensors can generate a signal indicating a threshold of stomach expansion. The sensor can be configured to measure positive and negative pressure changes which may indicate an increase or decrease in stomach size. A recording device may be used to record and transmit sensed data from the implant to the outside of the patient.

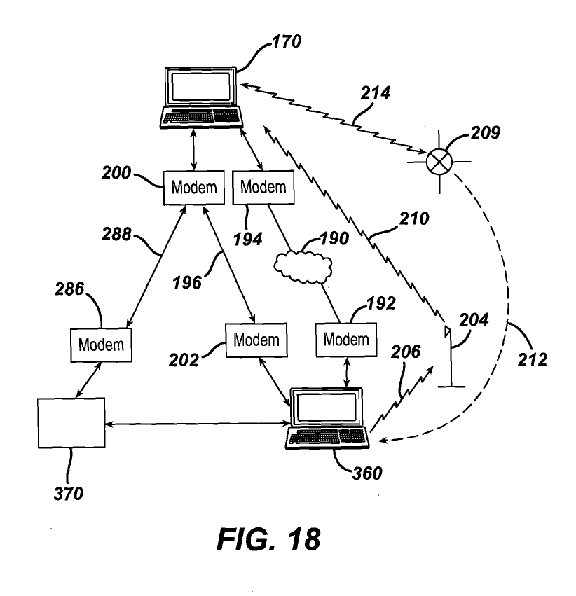

FIG. 17 illustrates yet another embodiment for adjusting a gastric coil device. In this embodiment, a plurality of rigid links 105 are rotatably coupled by a plurality of pivot pins 106. At either end of the coil are fixation segments 115 that prevent the passage of the device through either the esophagus or pylorus. Each fixation segment 115 may be comprised of a single deformable material and geometry that could be selectively straightened for insertion, but could be reshaped after insertion as shown to prevent migration of the coil out of the gastric cavity. Methods for inserting a coil with device features to prevent migration have been previously described. Alternatively, fixation segments 115 may also be comprised from a series of rigid links 105. Rigid links 105 comprising fixation segments 115 may be tethered to separate regions of the device so as to prevent migration. Methods for deploying a coil that requires tethers to establish fixation segments 115 are described in commonly owned and pending U.S. patent application Ser. No. 12/147,984, filed on Jun. 27, 2008 and pending U.S. patent application Ser. No. 12/163,009, filed on Jun. 27, 2008, which are hereby incorporated herein by reference in their entirety. As illustrated in FIG. 18, a lumen 107 preferably runs through the entire length of the device. Inside the lumen 107, an elongated balloon 108 is placed. The balloon is preferably made out of a material with a geometry that deforms under moderate pressures exerting loads along at least a portion of lumen 107. Balloon 108 should be able to maintain the desired pressure over time, should not degrade due to exposure to the gastric environment, and should be constructed of a material that is biocompatible. Examples of candidate materials include but are not limited to silicone, polyether polyester urethane, polyether polyester copolymer, and polypropylene oxide. The balloon is in fluid communication with a fill port 109. Numerous fill ports configurations for devices such as laparoscopic adjustable gastric bands are well known in the art and one skilled in the art would be able to adapt these for use in this application. Additionally, the sensor 102 could be in fluid communication with the balloon 108. Examples of sensors 102 that could be used in this application are described in commonly owned and pending U.S. patent application Ser. No. 11/065,410, filed on Feb. 24, 2005, pending U.S. patent application Ser. No. 11/369,682, filed on Mar. 7, 2006, pending U.S. patent application Ser. No. 11/369,389, filed on Mar. 7, 2006, pending U.S. patent application Ser. No. 11/369,531, filed on Mar. 7, 2006, pending U.S. patent application Ser. No. 11/668,122, filed on Jan. 29, 2007, pending U.S. patent application Ser. No. 11/673,642, filed on Feb. 12, 2007, and pending U.S. patent application Ser. No. 11/739,778, filed on Apr. 25, 2007, which are hereby incorporated herein by reference in their entirety. Although shown attached to an end of the coil, sensor 102 and fill port 109 could be housed in central section 117 while maintaining fluid communication with one or more balloons and without increasing the diameter of central section 117 which could hinder insertion and/or removal of the device.

After insertion into the gastric cavity, the fill port 109 is accessed endoscopically in order to inject or withdraw fluid from balloon 108. In one embodiment, fill port 109 is releasably connected to a fluid delivery device outside of the patient prior to delivery of the device into the gastric cavity. Balloon 108 may be partially filled prior to introduction into the patient, but not to an extent that would hinder insertion. Once placed in the gastric cavity, balloon 108 may be filled to the desired pressure. After balloon 108 is filled, the fluid delivery device is disconnected from fill port 109.

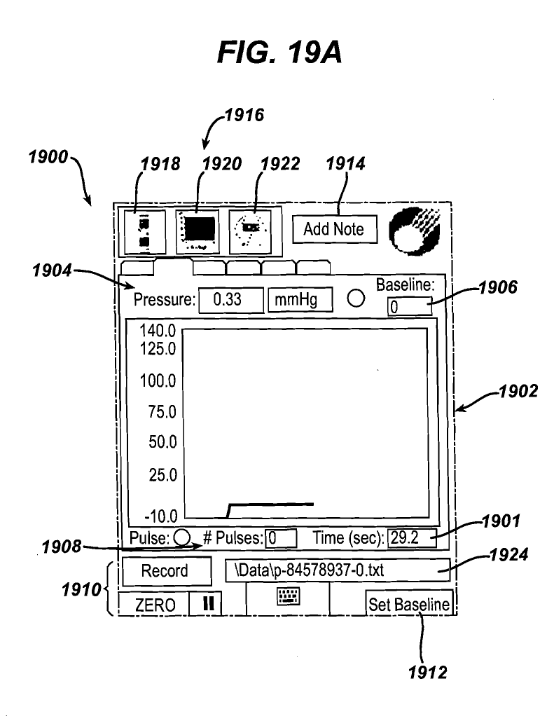

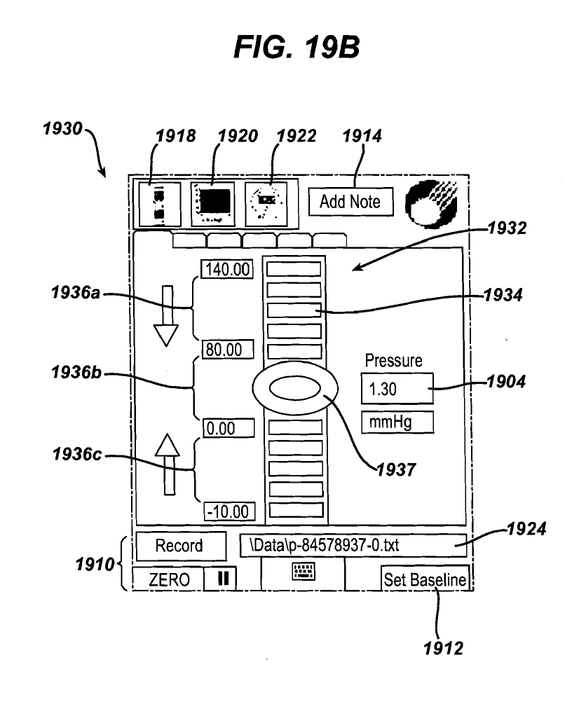

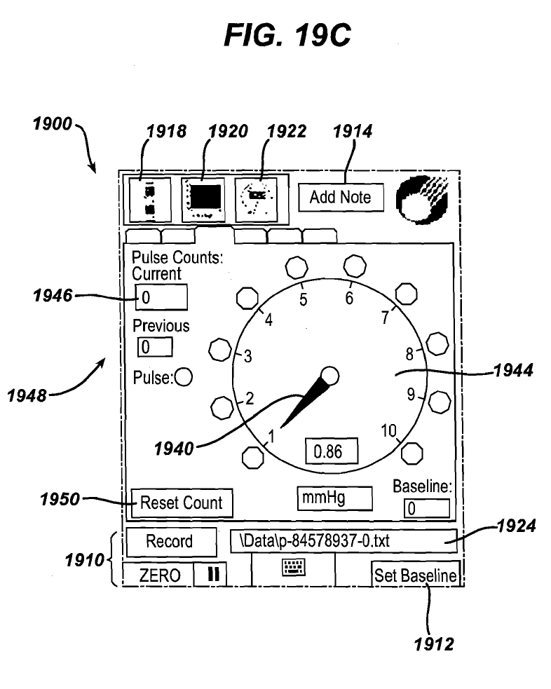

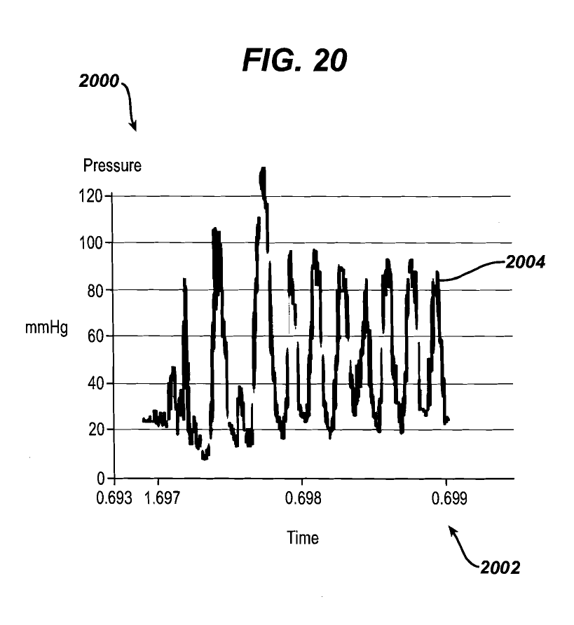

FIGS. 19 and 20 are close-up views showing details of individual links 105. As previously described, the external surfaces of the coil are ideally shaped to minimize or eliminate acute (pinch points) or long term (erosions) trauma to the gastric wall.

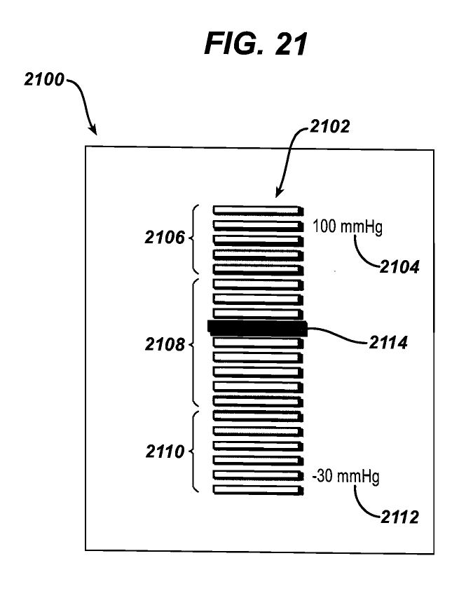

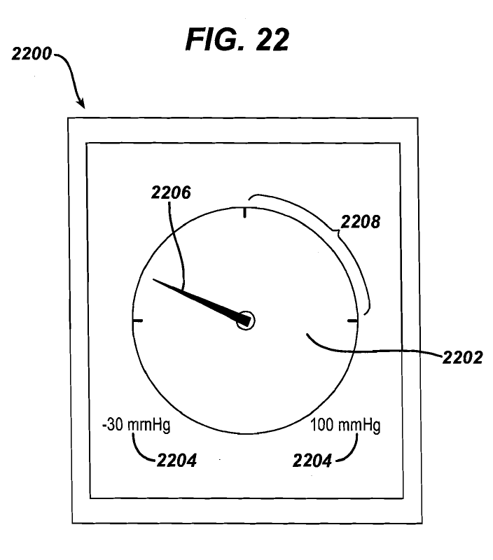

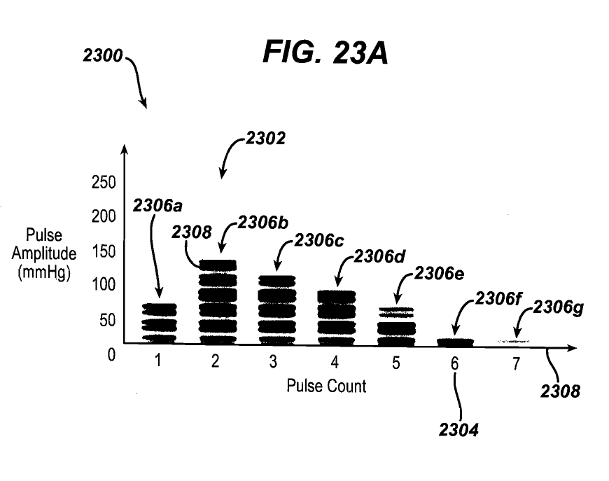

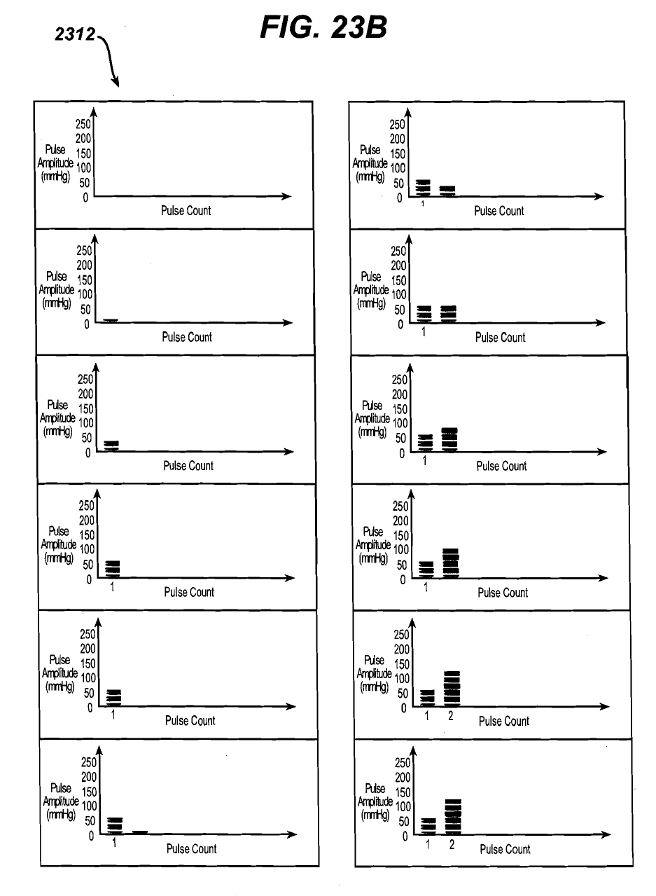

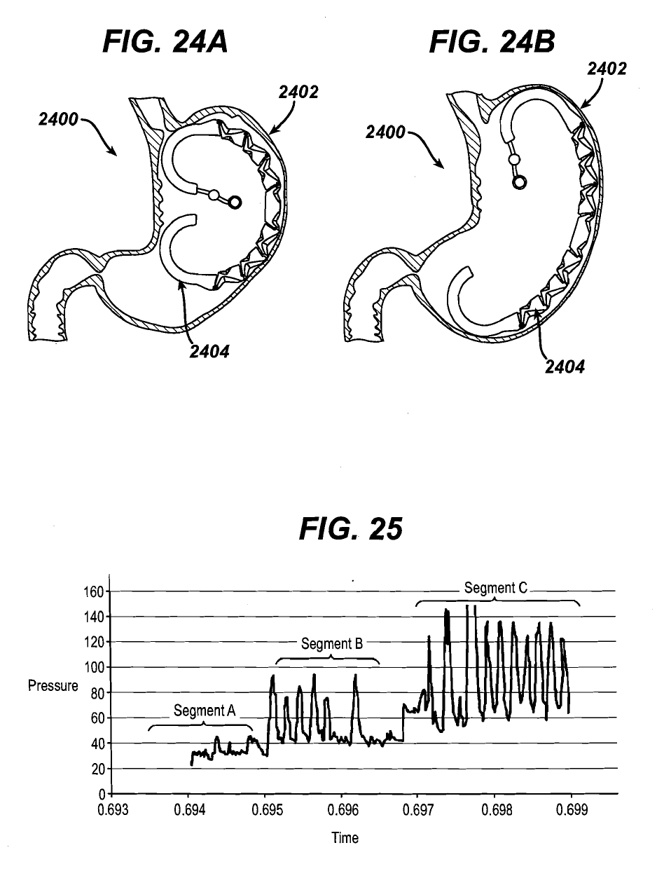

FIG. 21 is a cross-sectional view highlighting details of lumen 107. The lumen 107 is widened at both ends of each individual link in area 110 and 111. These widened areas 110 and 111 abut each other adjacent to pivot pin 106. As fluid is injected into port 109 and balloon 108 fills, the balloon 108 expands into widened areas 110 and 111 as illustrated by bulge 112 in FIG. 22.

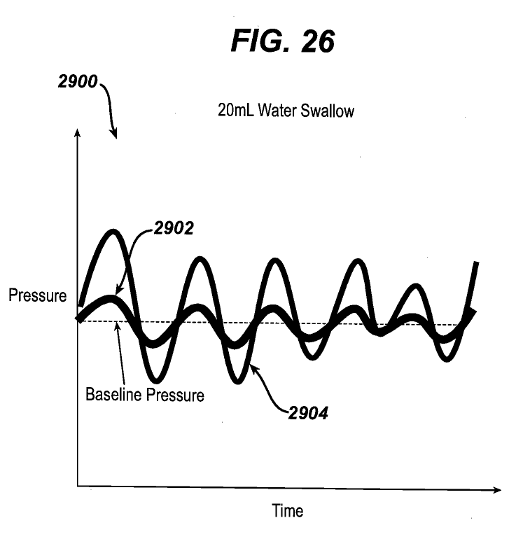

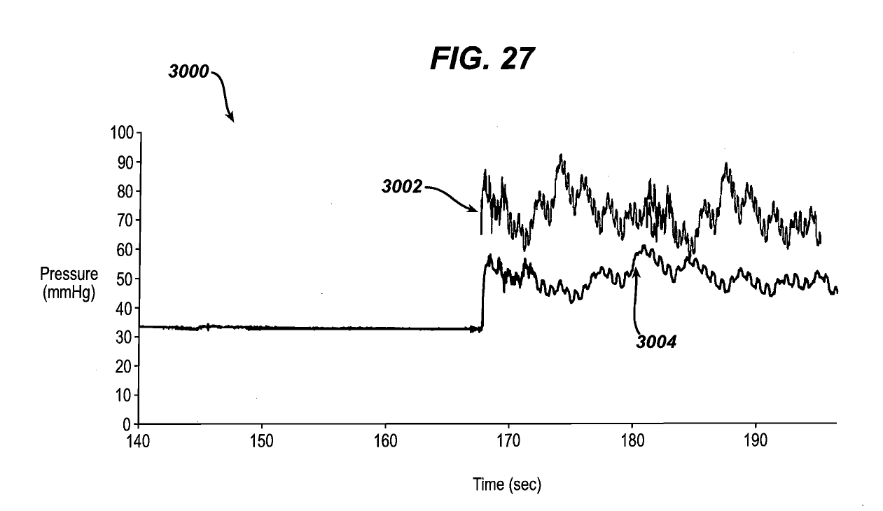

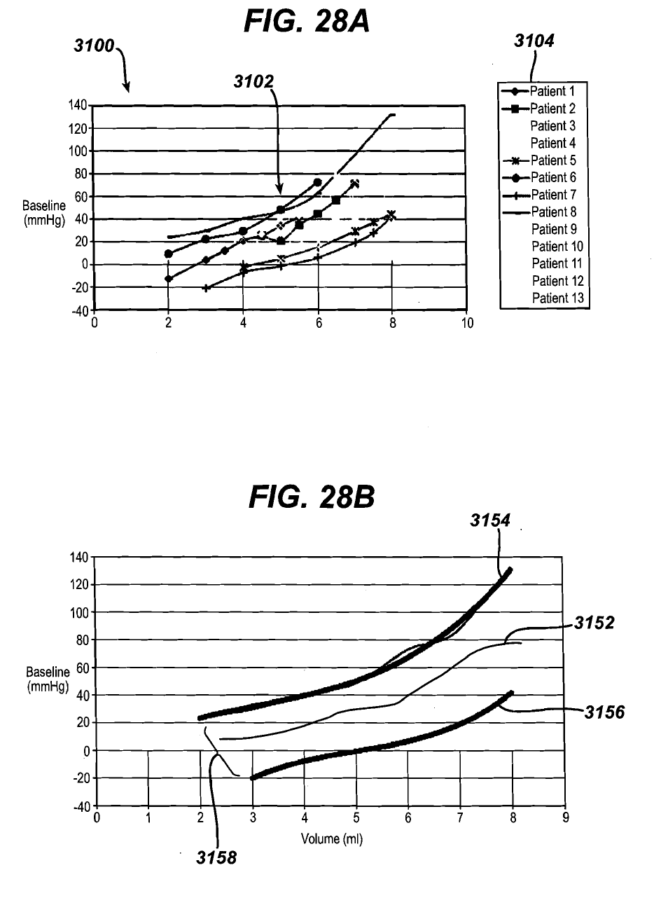

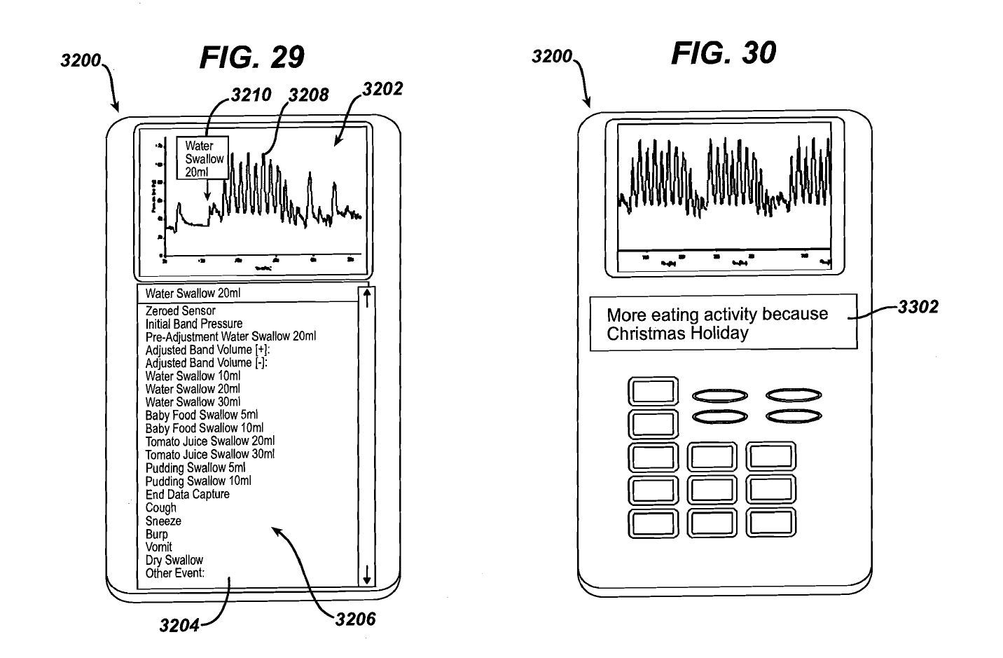

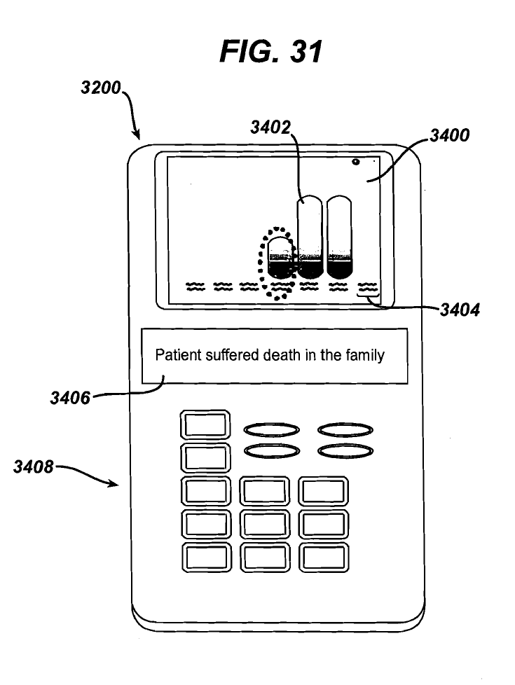

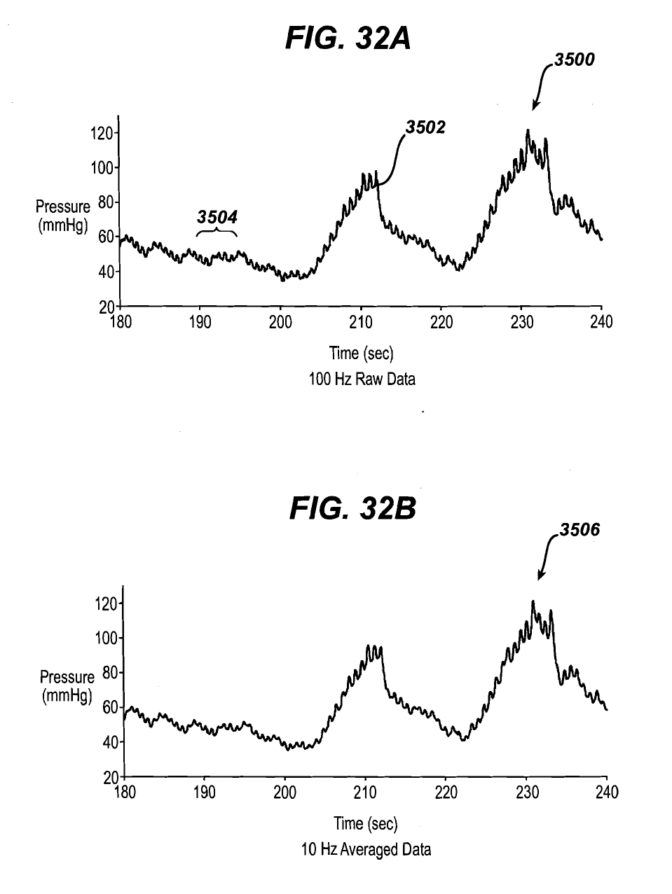

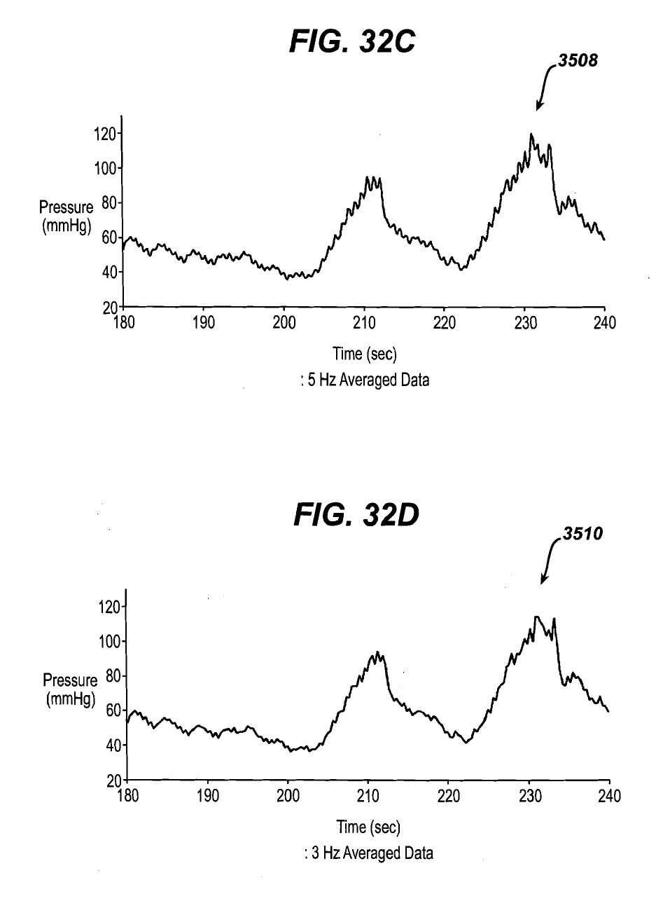

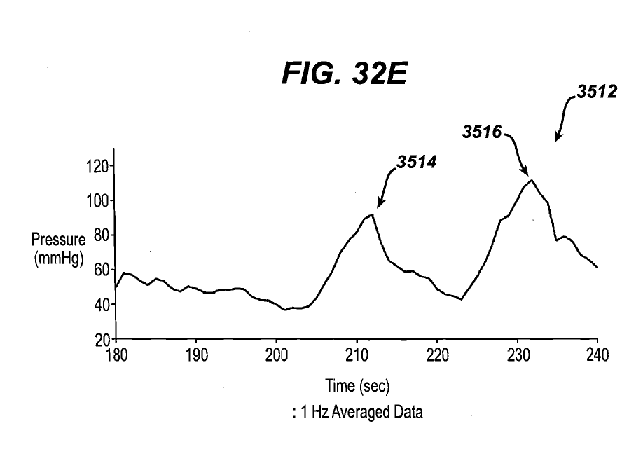

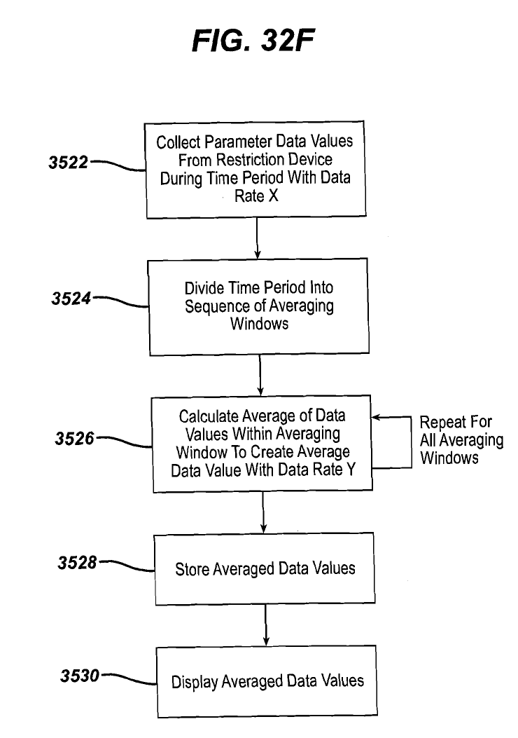

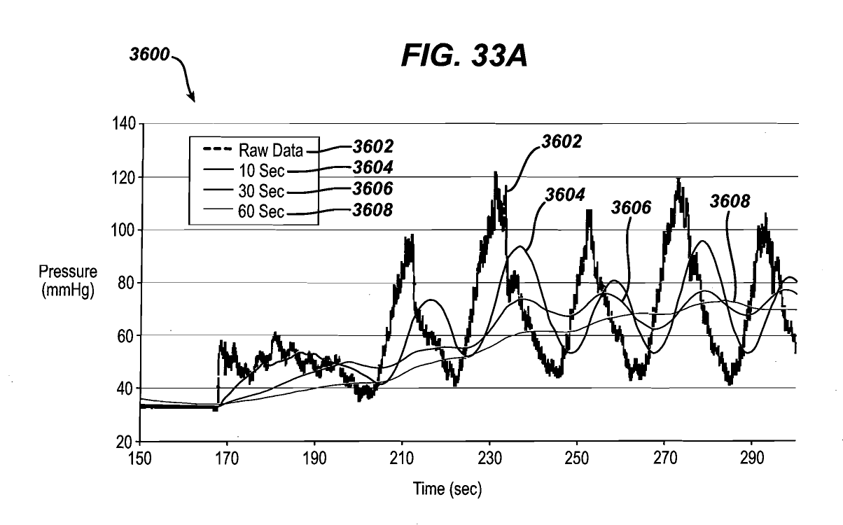

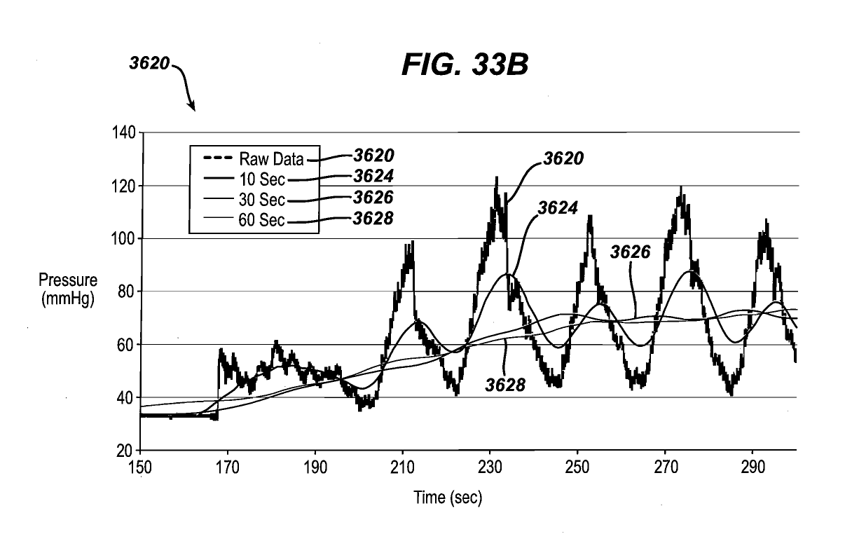

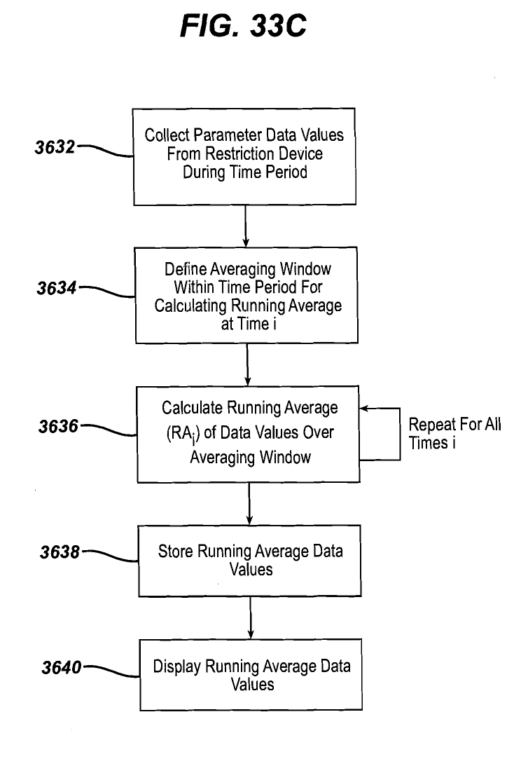

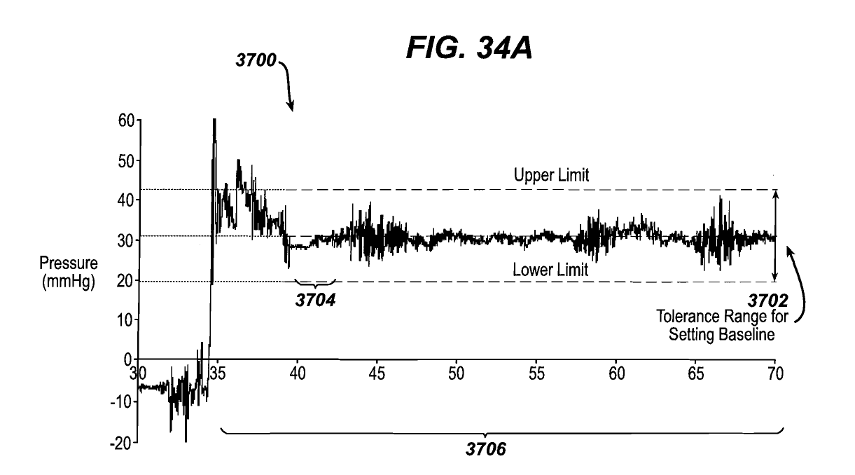

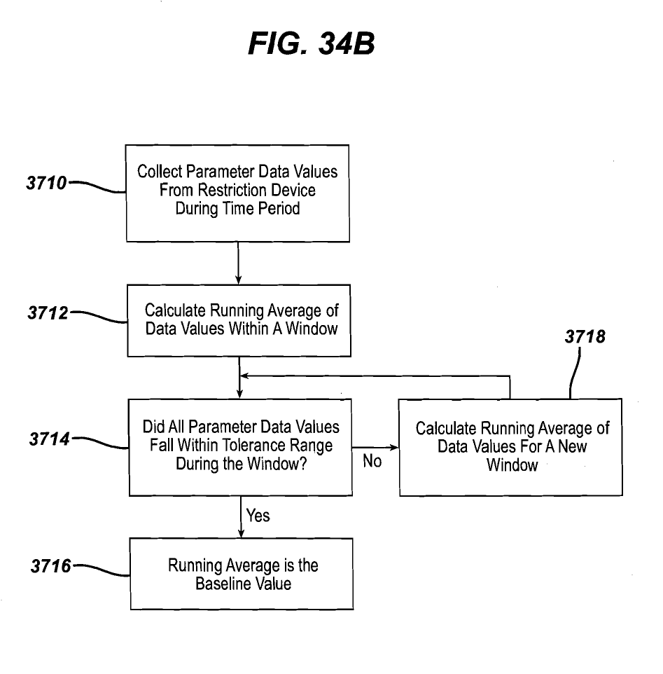

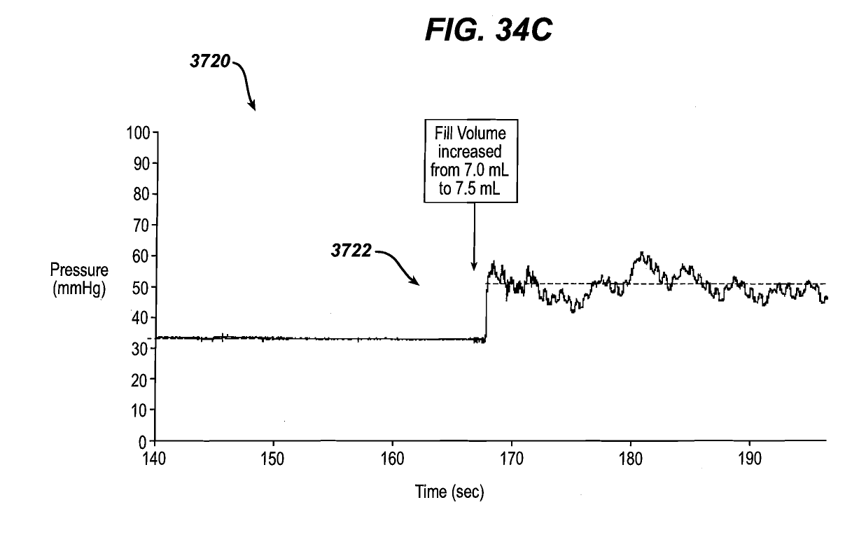

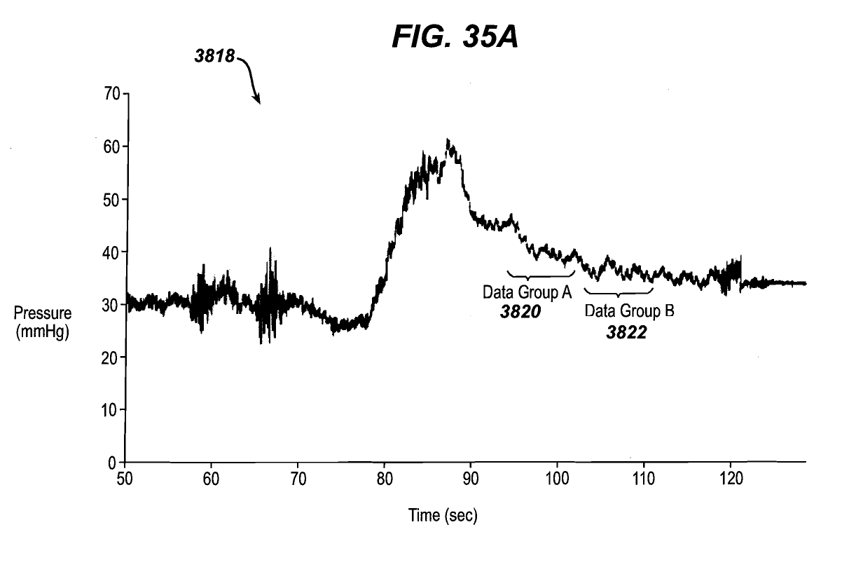

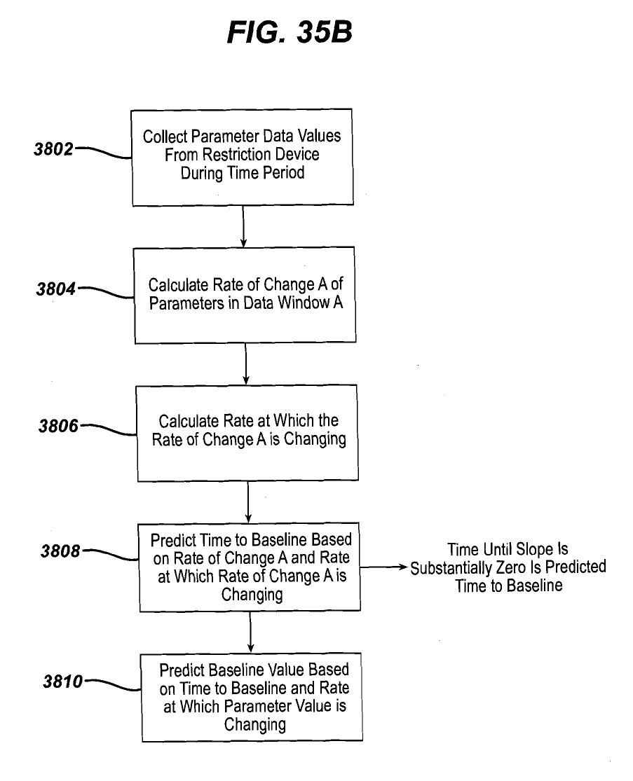

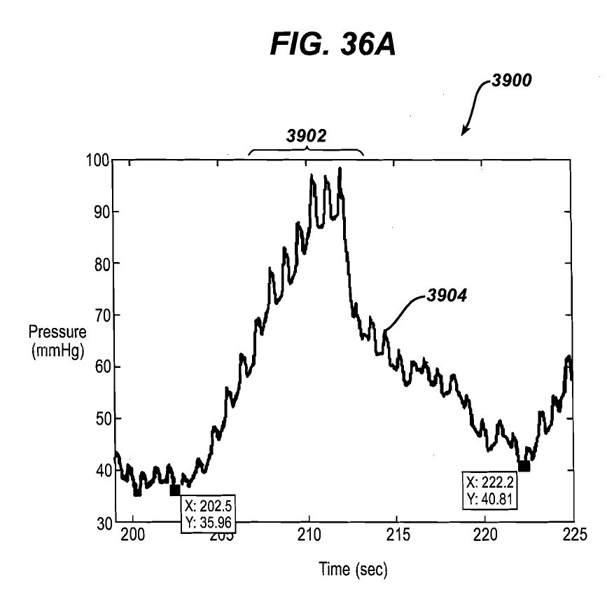

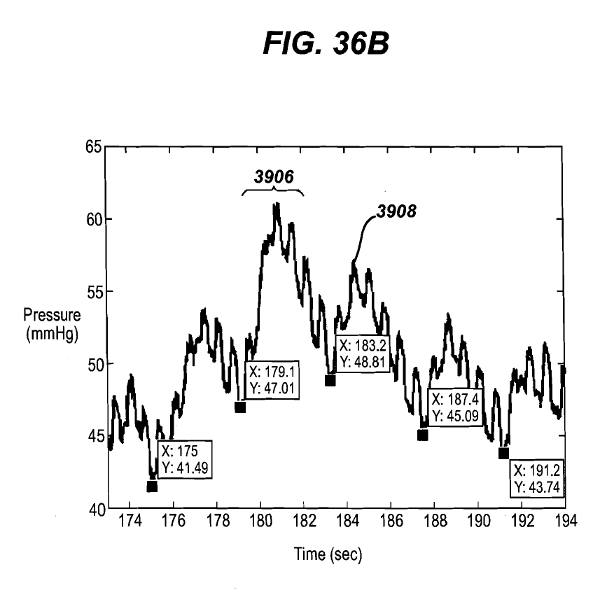

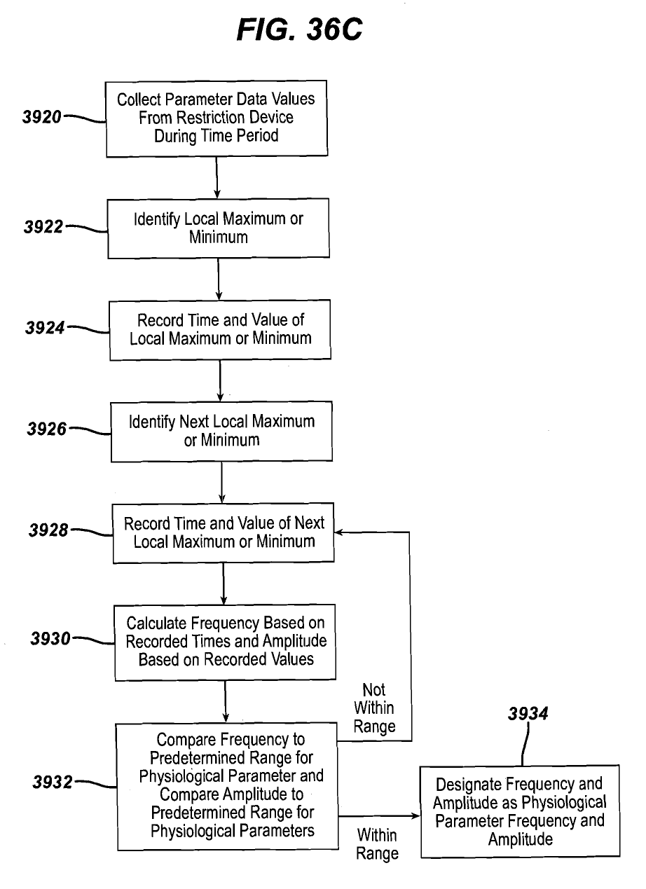

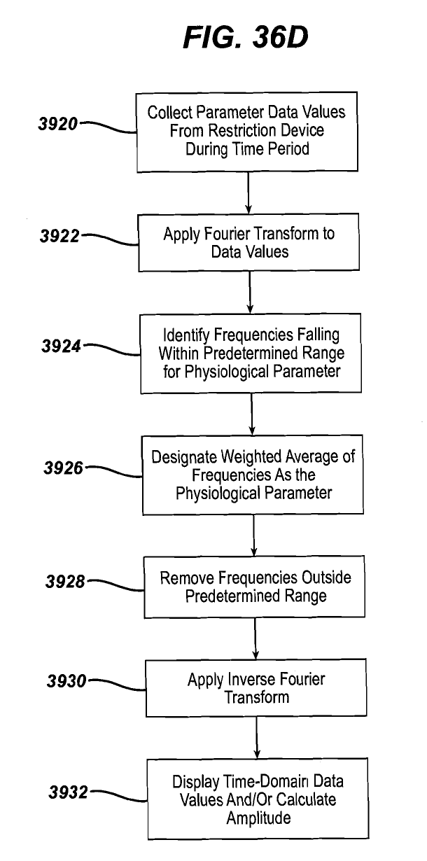

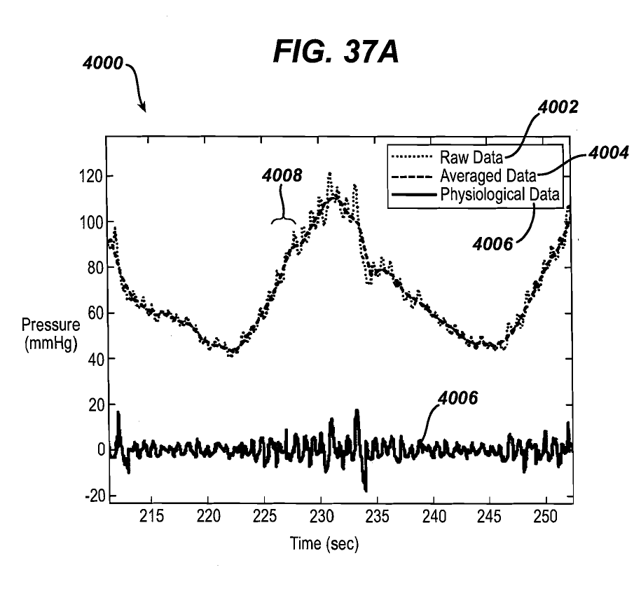

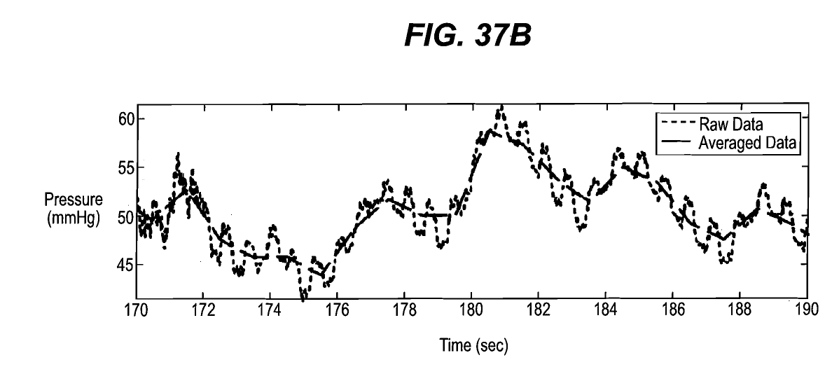

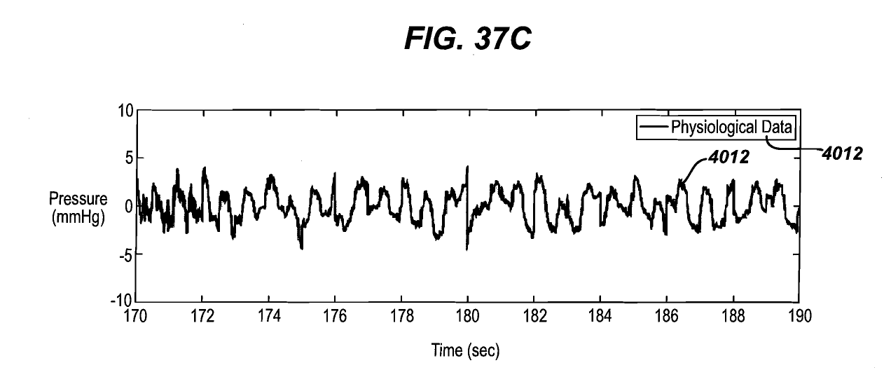

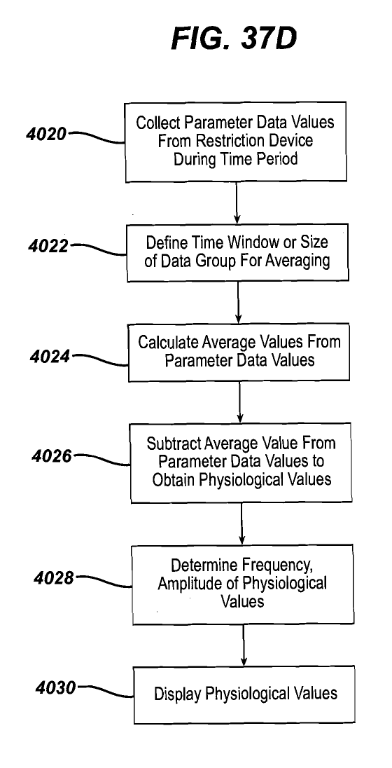

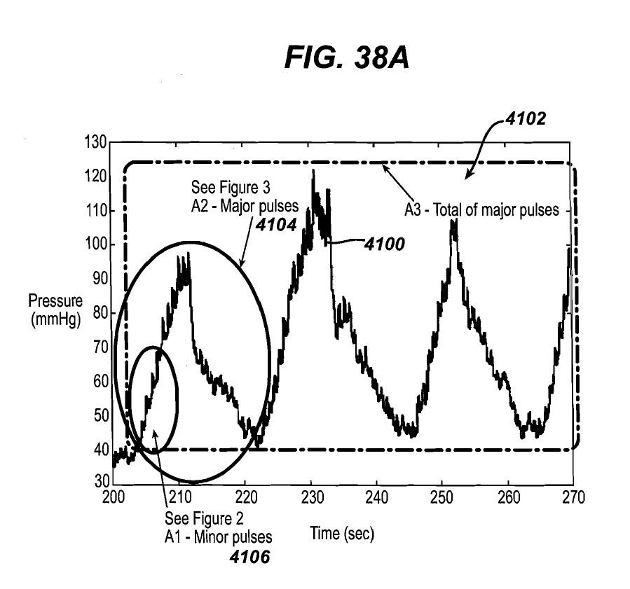

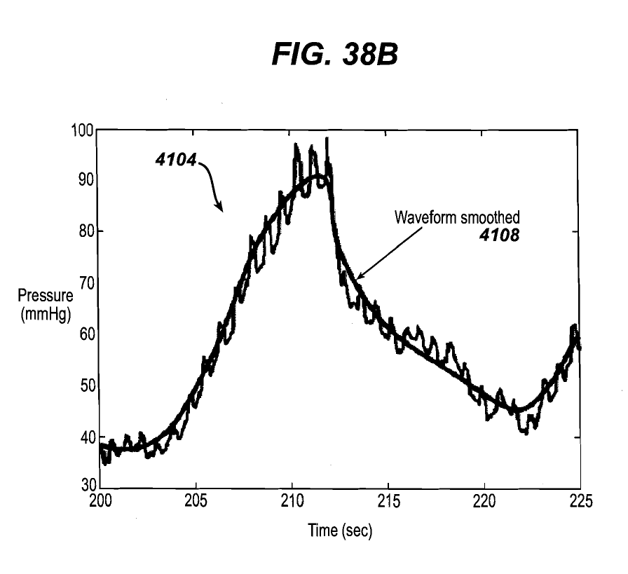

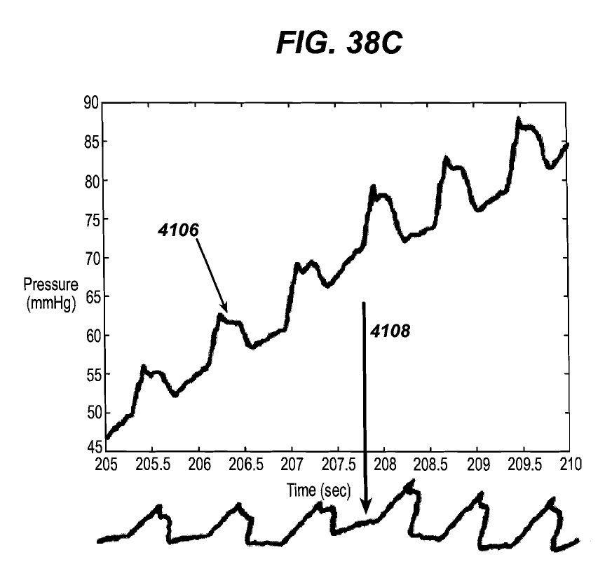

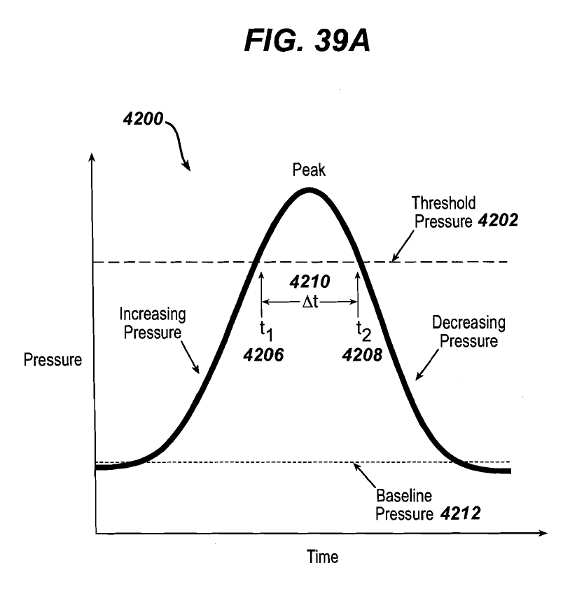

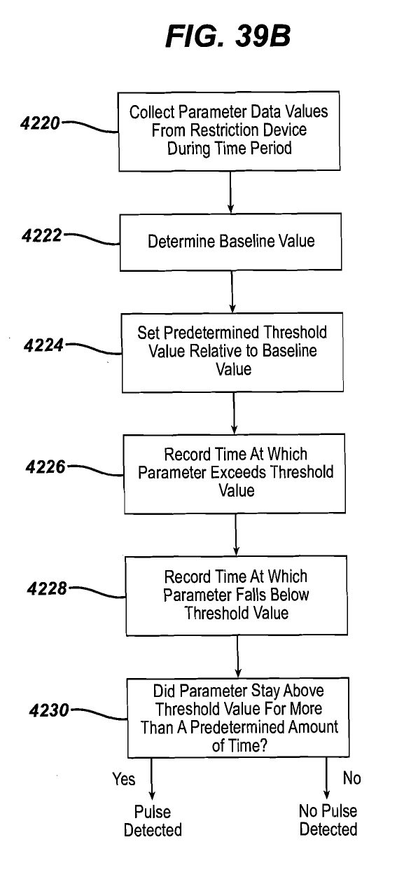

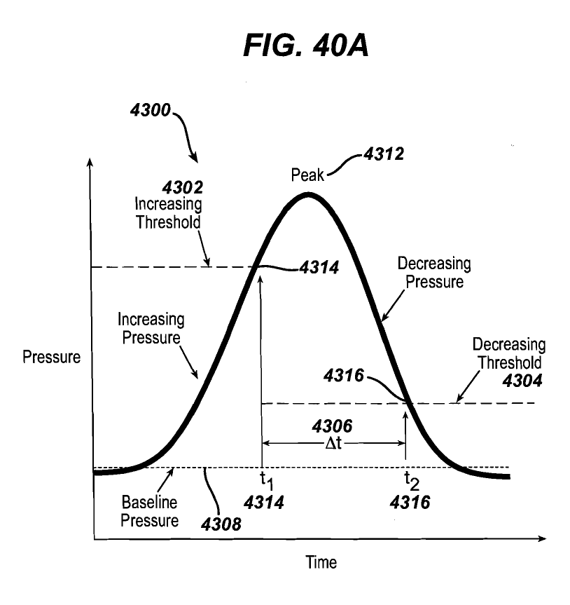

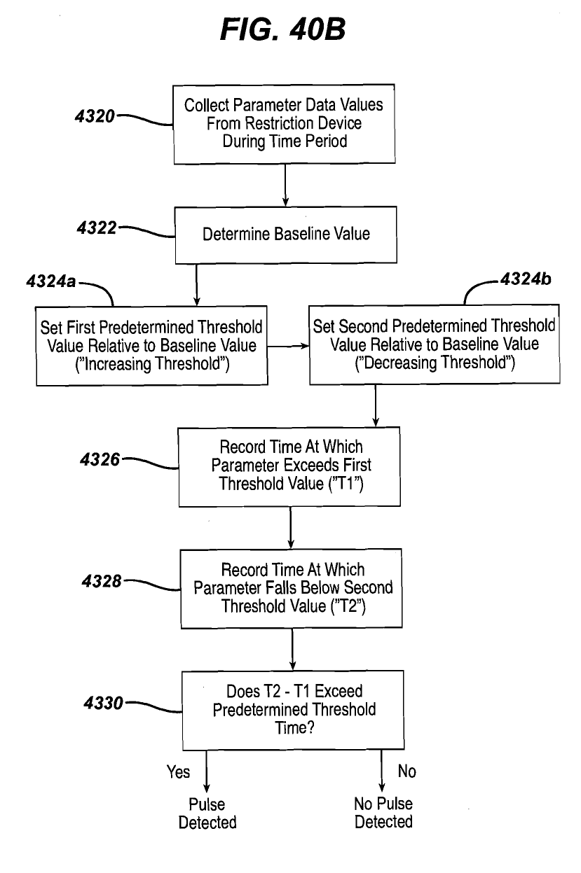

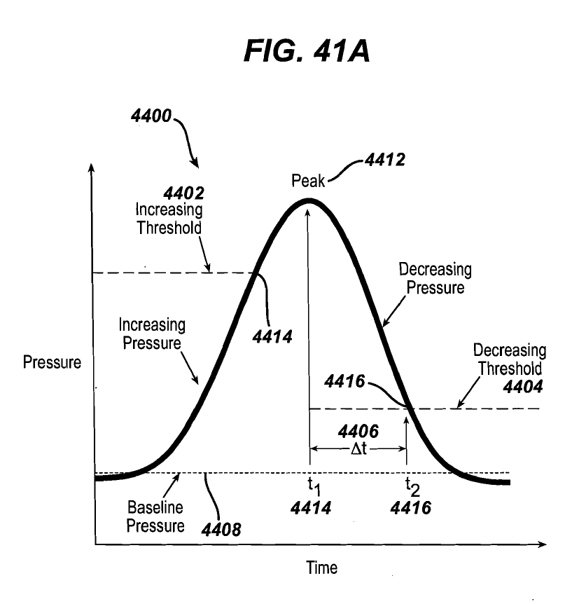

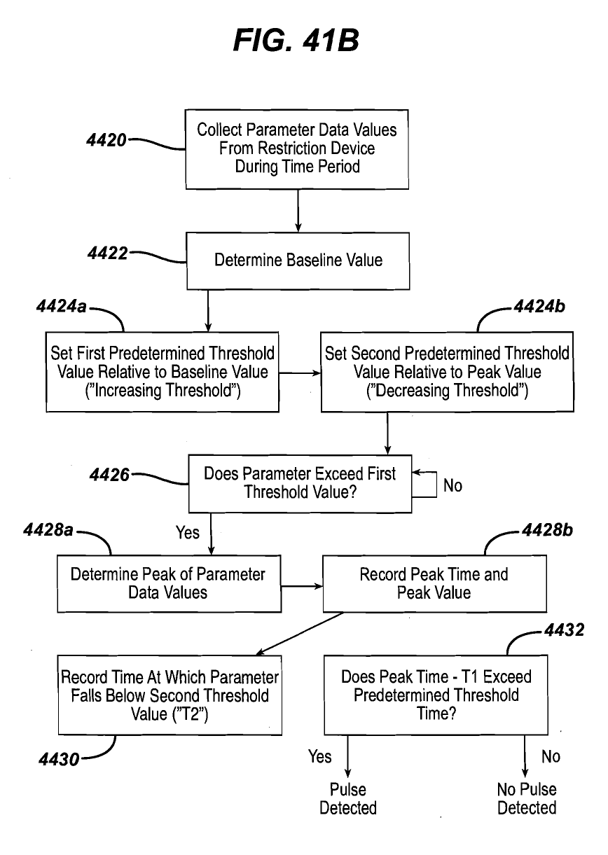

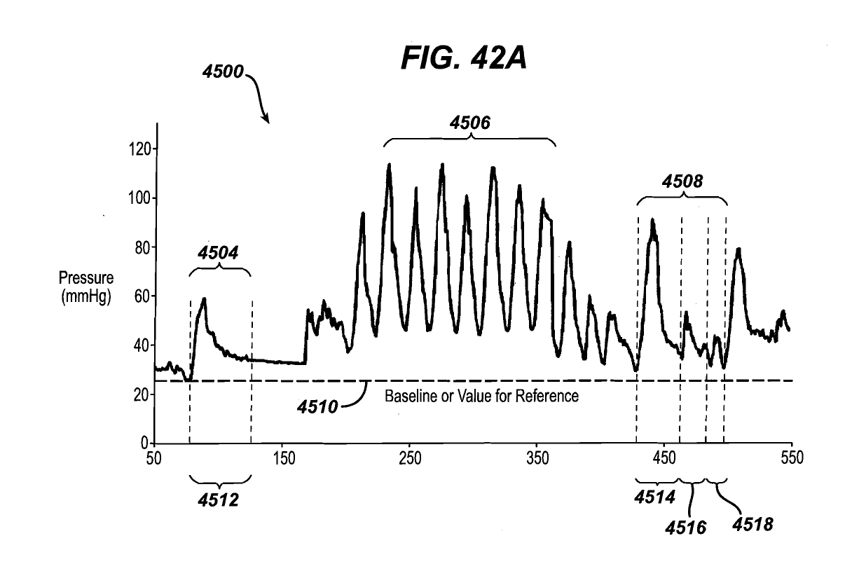

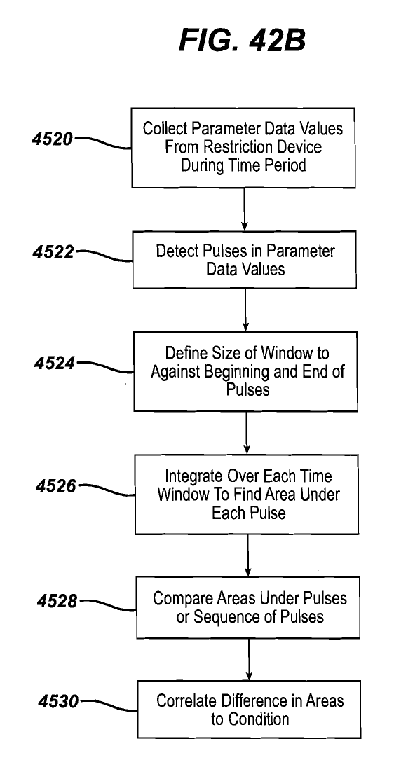

Continued filling of balloon 108 creates opposing forces on widened areas 110 and 111 of lumen 108. The force provides for a rotational force of individual links 105 about pivot pin 106, expanding the coil as illustrated in Fig. The expanded coil places an outward force on gastric cavity 46, flattening the gastric lumen 113 as illustrated in FIG. 25. As previously described, this distension of the stomach may trigger one of many mechanisms within the metabolic system (e.g., engaging natural stretch receptors of the stomach sooner, etc.), sending an early sense of satiation and/or a prolonged sense of satiety to the patient. As discussed above, minor alterations may be made to this embodiment so that the addition of fluid serves to reduce the overall shape of the coil rather than increase it.

In another embodiment the in lumen of the coil is at least partially filled with an expandable material such as a hydrogel such that the band can be inserted endoscopically in a substantially flat configuration. Once deployed into the stomach could automatically expand to a predetermined size by at least partially exposing the internal hydrogel to stomach fluids. Alternatively, the coil can be of a hermetically sealed construction where the hydrogel is expanded through a fluid port. The hydrogel could saturate at a given fluid absorption level thereby allowing the addition or removal of fluid to expand or contract the coil without the hydrogel further expanding.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present invention.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, ethylene oxide (EtO) gas, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam, etc.

Any patent, publication, application or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device, including an implant for placement within a hollow body organ, said device comprising:
a. a member having an undeployed shape for delivery within a hollow body and one or more deployed shapes for implantation therein;
b. said member having sufficient rigidity in its deployed shape to exert an outward force against an interior of the hollow body so as to bring together two substantially opposing surfaces of said hollow body; and
c. a means for changing the deployed shape of said member while implanted within said hollow body.

2. The device of claim 1 wherein said means for changing the deployed shape of said member is at least partially external to said hollow body.

3. The device of claim 1 wherein said means for changing the deployed shape of said member comprises a hydraulic means for adding or removing fluid from said member.

4. The device of claim 1 wherein said means for changing the deployed shape of said member comprises a mechanical means for applying a load to said member.

5. The device of claim 4 wherein said mechanical means for applying tension to said member further comprises a rotation member.

6. The device of claim 4 wherein said mechanical means for applying tension to said member further comprises a rotation member comprising a percutaneous implanted rotating control knob.

7. The device of claim 6 wherein said member comprises a series of connected individual links, said member having an internal lumen with a balloon therein.

8. The device of claim 7 wherein said member includes individual and distinguishable loading members, each of which is severable to change said deployed shape.

9. The device of claim 7 wherein said member includes individual and distinguishable loading members, each of which is dissolvable to change said deployed shape.

10. A device, including an implant for placement within a hollow body organ, said device comprising:
a. a member having an undeployed shape for delivery within a hollow body and one or more deployed shapes for implantation therein;
b. said member having sufficient rigidity in its deployed shape to exert an outward force against an interior of the hollow body so as to bring together two substantially opposing surfaces of said hollow body; and
c. a means for changing the deployed shape of said member while implanted within said hollow body wherein said means for changing the deployed shape of said member while implanted within said hollow body changes said deployed shape as a function of time.

11. The device of claim 10 wherein said means for changing the deployed shape of said member is at least partially external to said hollow body.

12. The device of claim 10 wherein said means for changing the deployed shape of said member comprises a hydraulic means for adding or removing fluid from said member.

13. The device of claim 10 wherein said means for changing the deployed shape of said member comprises a mechanical means for applying a load to said member.
